(19) United States
(12) Patent Application Publication
(10) Pub. No.: US 2008/0004679 A1
(43) Pub. Date: Jan. 3, 2008

(54) NON-INVASIVE MODULATION OF THE AUTONOMIC NERVOUS SYSTEM

(75) Inventors: Morteza Naghavi, Houston, TX (US); Mortaza Mark Naghavi, Houston, TX (US); Mojtaba Frank Naghavi, Houston, TX (US); Mark C. Johnson, Houston, TX (US); Timothy J. O'Brien, Anoka, MN (US)

Correspondence Address: WONG, CABELLO, LUTSCH, RUTHERFORD & BRUCCULERI,;L.L.P., 20333 SH 249, SUITE 600, HOUSTON, TX, 77070, US

(73) Assignee: THERMARX, Houston, TX (US)

(21) Appl. No.: 11/779,543

(22) Filed: Jul. 18, 2007

Related U.S. Application Data
(63) Continuation-in-part of application No. 11/425,919, filed on Jun. 22, 2006.
(60) Provisional application No. 60/693,122, filed on Jun. 23, 2005.

(51) Int. Cl. A61F 7/08

(52) U.S. Cl. 607/108

(57) ABSTRACT

The present invention is directed to methods and apparatus for modulation of the sympathetic-parasympathetic balance by application of heat, carotid and/or ocular message to reduce sympathetic tone or increase parasympathetic tone in a target muscle system to relieve a symptom of urinary hesitancy, shy bladder syndrome, DESD, urinary retention, or laryngeal spasm, as well as to monitor the efficacy of treatments for bladder conditions and to assist in the passage of medical devices through bodily sphincters as well as to treat congestive heart failure.

### CROSS REFERENCE TO RELATED APPLICATIONS

This application is a continuation-in-part of and claims priority to U.S. patent application Ser. No. 11/425,919, filed Jun. 22, 2006 and published as US2006/0293719 A1 on Dec. 28, 2006, which in turn claims priority based on U.S. Provisional Application Ser. No. 60/693,122 filed Jun. 23, 2005.

### FIELD OF THE INVENTION

This invention relates methods and apparatus for non-invasive modulation of the autonomic nervous system, in particular, the use of heat to modulate the balance between sympathetic and parasympathetic control of muscle tone.

### BACKGROUND OF THE INVENTION

Without limiting the scope of the invention, its background is described in connection with novel methods and apparatus for modulation of autonomic system control of muscle activity. The autonomic nervous system controls many vital bodily systems, including the cardiovascular system, gastrointestinal, urinary and bowel functions, temperature regulation, and muscle tone.

The autonomic nervous system is primarily composed of the sympathetic and parasympathetic systems. In certain diseases and conditions, the balance between sympathetic and parasympathetic system control is implicated either causally or in attempted remediation. For example, filling and emptying of the bladder, or “reflex micturition”, involves a balance of sympathetic and parasympathetic control. Filling of the bladder requires relaxation of the detrusor muscle of the bladder due to sympathetic stimulation of beta-adrenergic receptors as well as sympathetic stimulation of alpha-adrenergic receptors by norepinephrine causing contraction of the internal involuntary urethral sphincter of the bladder neck.

Conversely, emptying of the bladder is effected when the cortical center of the brain triggers inhibition of sympathetic impulses and stimulates the parasympathetic system to release acetylcholine which results in relaxation of the internal and external sphincters and contraction of the detrusor muscle. Changes to the physiology of the urinary tract as a consequence of aging and which affect continence include decreases in bladder elasticity with reduced bladder capacity resulting in more frequent urination and decrease in strength of the detrusor muscle, resulting in incomplete emptying.

Reflex micturition is implicated in Lower Urinary Tract Symptoms (LUTS), including those caused by prostatic enlargement or Benign Prostatic Hyperplasia (BPH). LUTS is quite common in men as they age. In one study of men aged 40-80, 54% needed to wake up at least once at night to urinate, 47% indicated they had terminal dribbling sometimes or frequently, 30% experienced urgency (although only 4% had urge incontinence), 21% experienced hesitancy, and 19% could retain urine in their bladder during the day for no more than 2 hours. Sladden M J et al. A Community Study of Lower Urinary Tract Symptoms in Older Men in Syndey, Australia, ANZ Journal of Surgery, May 2000, vol. 70, no. 5, pp. 322-328(7).

BPH is the most common benign neoplasm in men and can be identified histologically in half of all men at age 60, and in 90% by 85 years. The increase in size of the prostate inside its capsule exerts pressure on the urethra, which passes through the capsule, resulting in obstruction to urine flow. As the prostate enlarges, the gland is forced to press against the urethra in a clamp-like fashion. The bladder wall becomes thicker and irritable and is less extensible thus reducing capacity. The bladder can contract even when it contains small amounts of urine resulting in frequent urination. Ultimately, the bladder weakens and loses the ability to fully empty. Urine retention and strain on the bladder can lead to urinary tract infections, bladder or kidney damage, bladder stones, and incontinence. In the USA, about 25% of men will be treated for BPH by age 80, and over 300,000 surgical procedures are performed each year for BPH (mostly transurethral resection of the prostate, TURP).

In certain cases of hesitancy where the prostate is not significantly enlarged (no prostate disease), excessive sympathetic tone (due to anxiety and other factors) is present. One social anxiety syndrome resulting in inability to relax the urinary sphincter muscles is “paruresis” (a.k.a. shy bladder syndrome, bashful bladder syndrome (BBS), bashful kidneys, pee-phobia, urophobia, and psychogenic urinary retention) is characterized by difficulty in urinating in the presence of others. Individuals with BBS are sometimes referred to as paruretics. This syndrome may affect as much as seven percent (7%) of the US public, or 17 million people, both male and female, and affected individuals may be required to utilize self-catheterization in order to empty the bladder.

In individuals having hesitancy due to excessive sympathetic tone, diminishing the activity of the sympathetic nervous system by α-adrenoreceptor antagonists (alpha blockers) is helpful in treatment. The alpha blocker drugs aim at reducing sympathetic tone of the bladder particularly the neck of bladder. It can also help people with enlarged prostate in whom relaxing the tone of the neck can be useful. Alpha blockers that have been approved for the treatment of symptoms associated with BPH include the drugs terazosin (marketed as Hytrin), doxazosin (marketed as Cardura), tamsulosin (marketed as Flomax), and alfuzosin (marketed as Uroxatral). All of these drugs act to by relaxing the smooth muscle of the prostate and bladder neck to improve urine flow and to reduce bladder outlet obstruction. Terazosin and doxazosin were developed first to treat high blood pressure, while tamsulosin and alfuzosin were developed specifically to treat BPH. The problem with use of alpha blockers is that the entire body is treated to ameliorate a local condition. Each of these drugs can have use limiting side effects.

What are needed are methods and apparatus for modulating the balance between the sympathetic and parasympathetic components of the autonomic nervous system in the treatment of a number of conditions including by inducing a rapid anti-sympathetic effect that lasts only long enough alleviate conditions manifest by excessive sympathetic tone, including conditions such as hesitancy during urination and shy bladder.

### BRIEF SUMMARY OF THE INVENTION

The present invention is directed to methods and apparatus for use of heat to modulate autonomic system control of muscle activity. The controlled application of heat induces vasodilation by shifting the sympathetic-parasympathetic balance, including through the induced increase in local production of nitric oxide. Therefore, the tolerable heat therapy of the present invention is applied in conditions where increased production of nitric oxide is needed. In one embodiment of the invention methods and apparatus are provided for modulating an activity of a target muscle system in the body by applying heat to reduce sympathetic tone or increase parasympathetic tone in the target muscle system to relieve a symptom of urinary hesitancy, shy bladder syndrome, DESD, urinary retention, or laryngeal spasm. In another embodiment of the invention, the autonomic nervous system is modulated to decrease peripheral vascular resistance in the treatment of heart failure.

In one embodiment in the treatment of urinary hesitancy, heat is used for rapid transient increase in the activity of parasympathetic nervous system. Transient inhibition of sympathetic nervous system activity by heat is effected by application of heat locally (perineal) or systemically specially in areas with maximum sympathetic innervation such as the fingertips.

In one embodiment, the application of heat is short term and designed for acute effects in reduction of urinary hesitancy and increasing urinary flow by inducing muscle relaxation through exposure of the skin to tolerable heat to induce muscle relaxation and help with urination.

In another short term acute indication, the application of heat is designed for acute effects in treatment of urinary retention following catheterization or surgery whereby sphincter relaxation and detrusor contraction is induced through exposure of the skin to tolerable heat to help with urination. In other embodiments, heat is provided by diathermy.

In other embodiments, heat is utilized in conjunction with medical procedures involving the passage of instruments through sphincters and facilitates such passage by dilating the sphincter through a heat induced parasympathetic response induced by local or regional heating.

In certain embodiments, the heat is applied proximal or close to the target muscle system while in other embodiments the heat is applied regionally or even distally to create a systemic anti-sympathetic effect including an effect in the target muscle system. In certain embodiments, the heat is provided by a wearable appliance which may include heatable or heating inserts, pads and patches that are dimensioned for placement on desired anatomical locations including appliances disposed in, or in association with garments including underwear, garter type belts, gloves, socks, shoes, head and ear bands, caps, nose heaters, face masks, helmets, scarves, jackets, vests and full or partial body suits. In other embodiments, heating ear patches, ear rings, and/or nail covers are provided that include an adhesive for attachment and in which heat is generated chemically. In certain embodiments, the apparatus includes a heating element, a heating controller connected to the heating element, and a source of power for the heating element. Alternatively heat is provided by chemical reaction or stimulation of condusive materials by microwave radiation.

In other embodiment, the heat is provided by a non-wearable fixed or portable appliance such as a plumbing or bathroom fixture for delivering heat locally, regionally and/or distally to the target muscle system. The appliance may be adapted to deliver radiant heat, warm air, a warm hand hold, and heat through heating wraps, pads and blankets.

In one embodiment, specially adapted heating pads are provided in an essentially U or horse-shoe shaped configuration such that heat can be delivered to the lower abdomen and the ventral surface of the upper thighs while providing access to the genital region. Such specially adapted heating pads are applicable for personal use by conforming to the upper legs of a person seated on a toilet or situated over a bed pan. Alternatively, such heating pads are suitable for use in conjunction with certain medical procedures where autonomic relaxation affecting the organs and tissues of the internal lower abdomen would facilitate the comfort and success of the medical procedure, including for example, insertion of catheters, speculum etc.

Optionally, the heating can be used in conjunction with a pharmacologic approach such as treatment with drugs including alpha blockers, beta blockers, ACE inhibitors, muscarinic receptor agonists, and combinations thereof. In other embodiments, heating is utilized in conjunction with one or more additional non-pharmacologic techniques including controlled audio/visual input, carotid massage, ocular message, and/or stimulation with electric, magnetic, and/or electromagnetic neuromodulating devices used to increase local ANS activity in the target muscle system.

In one embodiment, an apparatus is provided for controllably inducing a carotid sinus reflex that includes at least one pad dimensioned to apply compression to one or both carotid sinuses; and a band for holding the one or more pads against the one or more sinuses. The pad may optionally include a controllable heating element and/or a controllable vibrating element. In other embodiments, apparatus are provided for controllably providing ocular compression and thereby inducing a parasympathetic response for inducing smooth muscle relaxation. The apparatus includes a binocular housing and a pad situated in each housing and dimensioned to apply compression to both eye balls.

In other embodiments, methods and apparatus are provided for relaxing muscles of sphincters such as in the bladder neck by delivering sufficient intraluminal local heating to cause relaxation of the muscles. The intraluminal local heating may be provided by a catheter or cystoscope having at least one heatable section. Alternatively, a parasympathetic stimulus sufficient to cause bladder emptying is provided by implanting a device that is heats differentially in response to RF, electromagnetic, ultrasound or microwave radiation from an external source. By differentially, it is meant the device heats in response to the external stimulation to an extent exceeding the responsive heating of tissue.

In other embodiments, methods and apparatus are provided for evaluating sympathetic over-reactivity, for example through use of a multi-channel heat monitoring device that continuously probes heat at fingertips and/or toes before, during, and after a sympathetic stimulator. In other embodiments, methods and apparatus are provided for evaluating the efficacy of a treatment, including a drug therapy, for urinary hesitancy, as well as for evaluating the effects of treatments on diuresis and bladder function by determining a urinary output flow by volume over time.

In another embodiment of the invention, methods and apparatus are provided for use of heat to shift the sympathetic-parasympathetic balance, including through the induced increase in local production of nitric oxide, in order to induce vasodilation and reduced resistance to peripheral blood flow in the treatment of symptoms of heart failure. The heat is provided by a wearable appliance or garment such as underwear, gloves, socks, shoes, helmets, scarves, jackets, vests and body suits.

### BRIEF DESCRIPTION THE DRAWINGS

For a more complete understanding of the present invention, including features and advantages, reference is now made to the detailed description of the invention along with the accompanying figures:

FIGS. 1A and B illustrate embodiments of the invention including placement of a heating element on the perineum. In FIG. 1A, the device can be a independent of a garment. In FIG. 1B, the perineal heating element is shown as held in position by, or incorporated as part of, a garment.

FIGS. 2A-E depict various areas where heat can be locally and regionally applied for parasympathetic modulation of the bladder.

FIG. 3 depicts a urinary catheter having a heatable section for parasympathetic modulation of the sphincter of the bladder neck.

FIG. 4 depicts appliances for carotid and ocular message.

FIG. 5 depicts an example of system for determining urinary output flow by volume over time.

FIG. 6 depicts an example of a heating element dimensioned for controllably heating the lower abdomen.

FIG. 7 depicts an ambulatory shy bladder/BPH treatment package including heatable thigh pads and heatable frontal pocket.

FIGS. 8A and 8B depict embodiments of heatable whole and partial body suits wherein heating elements are affixed to the fabric of the garment.

FIG. 9 depicts an example of an ambulatory shy bladder/BPH treatment solution including heatable pocket inserts.

FIG. 10 depicts examples of heating wraps, adhesive pads and partial gloves.

FIG. 11 depicts examples of heating toe caps and combined toe cap-sole shoe inserts.

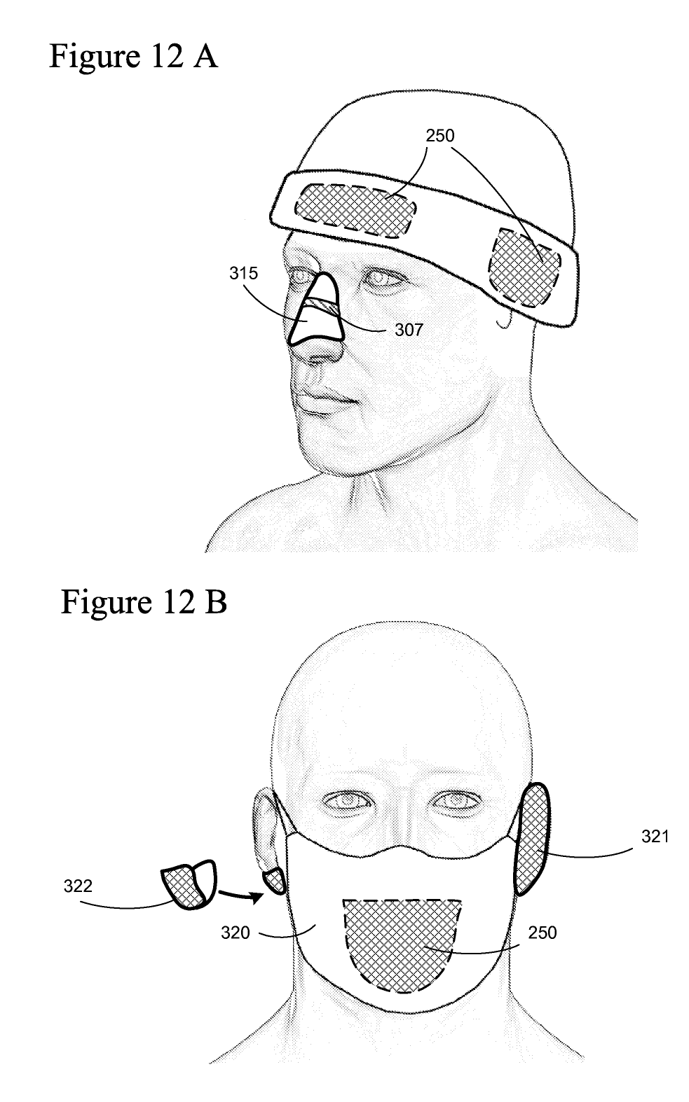

FIG. 12A depicts examples of heating nose patches, and head bands. FIG. 12B depicts a face mask heat exchanger as well as adhesive heating ear patches and ear lobe clips.

FIGS. 13A and B depict examples of heating wraps and adhesive pads.

FIG. 14 depicts one embodiment of a heating system for use in conjunction with a toilet.

FIG. 15 depicts an alternative embodiment of a heating system for use in conjunction with a toilet.

FIGS. 16A and B depict alternative embodiments of heating systems for use in conjunction with a toilet.

FIGS. 17A-D depict further embodiments of a toilet systems having heating components.

FIGS. 18A and B depict embodiments of heating bags adapted to provide parasympathetic stimulation.

FIG. 19 depicts an embodiment for deep tissue heating using ultrasound.

### DETAILED DESCRIPTION OF THE INVENTION

This invention discloses methods and means to reduce sympathetic stimulation or increase parasympathetic stimulation in order to modulate the activity and performance of desired smooth muscle cells in the body. The muscle cells can be in sphincters like the bladder neck sphincter, sphincter of the anus/rectum, and the ciliary sphincter in the eye. The muscle cells can also be the smooth muscle cells of the precapillary sphincter-like arterioles (a.k.a. arteriole sphincter band of smooth muscle at each capillary entrance) and the smooth muscle wall of luminal organs like the intestines.

As disclosed herein, methods and apparatus are provided for heating or increased local and/or regional warming to reduce sympathetic tone or increase parasympathetic tone as needed. In accordance with the invention, heat is used in at least two ways: in one embodiment local heat creates local anti-sympathetic effects partially mediated by nitric oxide, while in another embodiment heat is used to stimulate regional or systemic parasympathetic reflex responses such that heat applied distally creates an anti-sympathetic effect in a target tissue.

As disclosed herein, a heat may be combined with non-pharmacologic techniques for modulating ANS, mostly for regional and transient modulation based on anatomical reflex zones. These non-pharmacologic techniques may include non-invasive electric, magnetic, or electromagnetic neuromodulating devices used to increase local ANS activity. In other embodiments, pharmacologic approaches are combined with heating, the heating permitting lower dosages with reduced side effects.

While the making and using of various embodiments of the present invention are discussed in detail below, it should be appreciated that the present invention provides many applicable inventive concepts which can be employed in a wide variety of specific contexts in which modulating the sympathetic-parasympathetic balance is desired. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Abbreviations:

The following abbreviations are used throughout this application:
ACh Acetylcholine
ANS Autonomic Nervous System
BPH Benign Prostatic Hyperplasia
CSH Carotid Sinus Hypersensitivity
(L-NAME) N^{G}-nitro-L-arginine methyl ester (NO synthase inhibitor)
LUTS Lower Urinary Tract Symptoms
NO Nitric Oxide
NOS Nitric Oxide Synthetase
PAT paroxysmal atrial tachycardia
SBS Shy Bladder Syndrome,
SNP sodium nitroprusside

The phrase “urinary hesitancy” refers herein to difficulty commencing the flow of urine or slowed or delayed start of the urinary stream.

A “sphincter” is a circular muscle that normally maintains constriction of a natural body passage or orifice and is capable of relaxation as required for normal physiological functioning. There are a number of different sphincters in the human body, examples of which include: the two sphincters of the anus, the cardiac sphincter at the upper portion of the stomach, the pyloric sphincter at the lower end of the stomach, the urethral sphincter controlling emptying of bladder, the sphincter of Oddi (a.k.a. Glisson's sphincter), which controls secretions from the liver, pancreas and gall bladder into the duodenum and the ciliary sphincter in the eye. The phrase “precapillary sphincter” or “arteriole sphincter” refers to the band of smooth muscle at the junction of arterioles and capillaries and thus at each capillary entrance.

The phrase “diathermy” means the controlled production of deep heating beneath the skin in the subcutaneous tissues, deep muscles and joints for therapeutic purposes. Current diathermy devices on the market generate deep heating by using radio (high) frequency, microwave or ultrasonic energy. Current ultrasonic diathermy devices operate in a frequency range of 0.8 to 1 MH Z and generate heat by acoustic vibration. Radio frequency (r.f.) diathermy is assigned an operating frequency of 27.12 MH Z (short wave) by the Federal Communications Commission. Microwave diathermy is assigned 915 MH Z and 2450 MH Z as operating frequencies (these are also Microwave oven frequencies). The present informal position of the Food and Drug Administration is that a diathermy device should be capable of producing heat in tissue from a minimum of 104° F. to a maximum of 114° F. at a depth of two inches in not more than 20 minutes. RF heating can be done by dielectric or inductive methods and the physical configuration of the device is designed in accordance with electrical engineering principals depending on the ultrasound, MW or RF method desired.

As used herein, the term “wearable appliance” includes heatable inserts or pads that are dimensioned for placement in desired anatomical locations, including stand-alone appliances, appliances disposed in garments, and appliances that are used in association with a garment. Appliances that are used in association with a garment include appliances that are worn inside and those that are worn outside of the garment. Wearable appliances also include applicants that may be dimensioned to be carried, such as for example, a hand-warmer for a pocket. As used herein, the term “non-wearable” appliance includes fixtures and/or portable devices that may be placed in a bathroom or facility but are not dimensioned to be attached or carried by an individual during ambulation.

As used herein, “proximal” means nearest or closest to. Thus, areas and tissues proximal to the bladder include the perineum and pubic areas in addition to the detrusor muscles and the muscles of the bladder neck. Regional or local means in the general vicinity. Thus, as used herein “local” heating of the bladder may include heating of areas proximal to the bladder and further may include heating in the groin, lower abdomen and upper inner thigh areas. “Distal” in the context of the present invention means the opposite of proximal and means at a distance apart from the structure described. Areas distal to the bladder include, for example but without limitation, the face, hands and feet.

As used herein, the phrase “when desired” means surrounding the time at which the relevant function is desired.

In response to variations in environmental phenomena including temperature, food intake, and stressful experiences, afferent nerves from both systems convey impulses from organs, muscles, the circulatory system and the periphery of the body to controlling centers in the medulla, pons and hypothalamus of the brain. From these centers, efferent impulses are conveyed to all parts of the body by the parasympathetic and sympathetic nerves. Typically, these responses are largely involuntary automatic or reflex responses.

Parasympathetic system impulses are conveyed through cranial nerves number 3, 7, 9, and 10 (vagus nerves), in addition to some sacral nerves. Sympathetic impulses are conveyed down the spinal cord to sympathetic nerve bodies (ganglia) alongside the spine from which impulses travel to other nerve bodies (or neurons) in pathways that are in conjunction with blood vessels. Both sympathetic and parasympathetic systems include two groups of motor neurons: preganglionic neurons, arising in the CNS, and running to ganglions where they connect via synapses to postganglionic neurons, which run to effector tissues and organs.

Acetylcholine (ACh) is the neurotransmitter of the preganglionic sympathetic neurons. Release of ACh stimulates action potentials in the postganglionic neurons, which ultimately release the neurotransmitter noradrenaline (a.k.a. norepinephrine). Noradrenaline is excitatory is some cases and inhibitory in others. Thus, the release of noradrenaline stimulates increase in heartbeat and blood pressure, dilation of pupils and trachea and bronchi, conversion of glycogen into glucose in the liver, shunting of blood from the skin and viscera to the skeletal muscles, brain, and heart, inhibition of gastrointestional peristalsis and inhibition of contraction of the bladder and rectum. In opposition to the effects of the sympathetic system, parasympathetic stimulation causes slowing down of the heartbeat, lowering of blood pressure, constriction of the pupils, increased blood flow to the skin and viscera, peristalsis of the GI tract and contraction of the bladder. As with the sympathetic system, ACh is also the neurotransmitter for presynaptic parasympathetic neurons. In contrast, ACh is also the neurotransmitter for many postganglionic parasympathetic neurons. However, some postganglionic neurons release nitric oxide (NO) as their neurotransmitter. In such neurons, NO is formed by the NOS mediated conversion of L-arginine to citrulline. Once produced, NO is a highly diffusible agent able to elicit effects relatively far from the site of production. As a consequence of diffusion, the source of NO is a determinant of its concentration and a major factor determining the biological effect. At low concentrations, the direct effects of NO predominate. The principal direct effect of NO is the activation of soluble guanylate cyclase to increase levels of cyclic guanosine 3′5′-monophosphate (cGMP). The cGMP acts as a second messenger to induce relaxation via modified activity of protein kinases, phosphodiesterases (PDEs) and ion-channels that regulate contractile protein activity.

Nitric oxide is the primary neurotransmitter responsible for smooth muscle relaxation in the lower urinary tract and is thus a critical mediator in regulating bladder function. Nitric oxide synthetase (NOS) containing parasympathetic neurons that cause relaxation upon stimulation densely populate the prostate, urethra and bladder. However, the density of NOS containing nerves is highest in the outlet region or neck of the bladder. During emptying of the bladder, or voiding, cholinergic parasympathetic nerves induce relaxation of the smooth muscle of the bladder neck and proximal urethra.

Nitric oxide (NO) has been shown to be involved in cutaneous active vasodilation induced by systemic application of heat on the basis that local inhibition of NO synthetase resulted in inhibition of cutaneous local perfusion while local perfusion of the NO donor, sodium nitroprusside, resulted in maximum local cutaneous perfusion. See D L Kellogg Jr., C G Crandall, Y Liu, N Charkoudian, and J M Johnson. “Nitric oxide and cutaneous active vasodilation during heat stress in humans” J Appl Physiol 85 (1998) 824-829. Similarly, it was found that NO mediates vasodiliation in response to local application of heat. See D L Kellogg Jr., Y Liu, I F Kosiba, and D. O'Donnell. “Role of nitric oxide in the vascular effects of local warming of the skin in humans” J Appl Physiol 86 (1999) 1185-1190. Conversely, local cooling induces cold-sensitive afferent nerves to activate sympathetic nerves to release norepinephrine, which leads to local cutaneous vasoconstriction. J M Johnson, T C Yen, K Zhao, and W A Kosiba. “Sympathetic, sensory, and nonneuronal contributions to the cutaneous vasoconstrictor response to local cooling” J Physiol Heart Circ Physiol 288 (2005) H1573-H1579.

In accordance with the present invention, heat is used as a treatment for rapid or transient reduction of sympathetic nervous system activity or increasing the activity of parasympathetic nervous system in various medical conditions where shifting the balance towards lower sympathetic nervous system activity is desired. The heat can be local or generalized (systemic), transient or long term.

In one embodiment of the invention, pharmacologic (drug) approaches are combined with heating. However, in this combination, the drugs expected to be effective at a lower dosages than they are given for their primary indication, thereby reducing side-effects. The alpha blocker drugs (α-adrenoreceptor antagonists), which reduce sympathetic tone of the bladder particularly the neck of bladder, may be particularly useful in combination with heat therapy. Alpha blockers including terazosin (marketed as Hytrin), doxazosin (marketed as Cardura), tamsulosin (marketed as Flomax), and alfuzosin (marketed as Uroxatral) are already approved for BPH. Other potentially useful drugs include angiotensin converting enzyme (ACE) inhibitors and beta-adrenergic receptor blocking agents (β blockers) such as propranolol (Inderol), which reduce excessive sympathetic activity.

Medications that produce the same effects as the parasympathetic nervous system are called “cholinergic drugs” because they produce the same effects as acetylcholine, which is the most common neurohormone of the parasympathetic nervous system. Examples of direct acting cholinergic drugs that are expected to favor parasympathetic type relaxation for purposes of relaxation of the neck of the bladder include agonists of muscarinic receptors, which are principally responsible for smooth muscle stimulation. Muscarinic receptor agonists that cause parasympathetic over activity include choline esters such as bethanechol chloride (Urecholine®, orally available agent with fewer cardiovascular effects and longer-half-life), arecholine (also an agonist of nicotinic receptors), carbachol (i.e. Miostat®, some nicotinic activity, typically used to treat glaucoma). Clinically available alkaloids that are direct muscarinic receptor agonists include pilocarpine (i.e. Akarpine®, muscarinic selective agent typically used to treat glaucoma).

Examples of indirectly acting cholinergic drugs that function as muscarinic receptor agonists (act by inhibiting acetylcholine esterase, thereby reducing the destruction of acetyl choline) include ambenonium chloride (Mytelase), edrophonium chloride (Tensilon), and piridogstimina (Mestinón), neostigmine (Prostigmine) and physostigmine. In one embodiment, topical sensory nerve blockade by an anesthetic cream may also be employed to encourage vasodilation and parasympathetic relaxation.

In another embodiment of the invention, heat treatment is combined with other non-pharmacologic methods for decreasing sympathetic tone and increasing parasympathetic tone including e.g. mental relaxation, ablation of light, sound and all other established methods. In one embodiment, the specialized heating devices are combined the training exercises such as yoga exercise and biofeedback. In other embodiments, sensory input is provided to the ANS including audio visual input such as via a head set or helmet that blocks sympathetic stimulating audiovisual input from the environment and creates instant relaxation. The head set can be adapted to provide relaxing audio input (including the sound of running water) and/or reduced light. Such a head set or helmet may be provided with heating capability specially for gentle heating the ears which are heavily innervated by sympathetic nerves. It is anticipated that individual requirements for attaining a desired level of ANS modulation will vary widely. Thus, a combination of approaches may be required for certain individuals with high adrenergic activity, including heating in addition to one or more of: controlled audio/visual input, pharmacologic treatment, and stimulation by electric, magnetic, or electromagnetic devices. In one embodiment, a whole or partial body suit is provided with multiple heating regions to modulate the ANS to reduce sympathetic activity. Patients with severe cases of ANS imbalance may benefit from using this on a chronic or periodical basis. It can be used an alternative for rapid reduction of anxiety in clinical settings. A combination with pharmacologic methods may be chosen by physicians or medical professional.

Further non-pharmacologic approaches for modulating ANS are combinable with the heat therapy disclosed herein. These approaches are mostly for regional and transient direct stimulation of the nervous system but also include systemic applications. These non-pharmacologic techniques may include electric, magnetic, or electromagnetic devices, including those adapted for local electrical or magnetic stimulation of desired nerves including the sacral nerve. Stimulation is modulated and tuned in the individual patient to increase parasympathetic rather than sympathetic responses in conjunction with heat therapy.

The following examples are included for the sake of completeness of disclosure and to illustrate the methods of making the compositions and composites of the present invention as well as to present certain characteristics of the compositions. In no way are these examples intended to limit the scope or teaching of this disclosure.

Parasympathetic Stimulation of the Urinary Tract:

In one embodiment of the invention, methods and apparatus are provided for using heat to stimulate the parasympathetic nervous system to release the neck of the bladder and permit emptying of the bladder for relief of urinary hesitancy, shy bladder syndrome, DESD, and urinary retention.

Efferent and afferent nervous pathways innervating the lower urinary tract include three sets of peripheral nerves, the sacral parasympathetic traveling through the pelvic plexus, the thoracolumbar sympathetic traveling through the sympathetic chain ganglia, and the sacral somatic or pudendal nerves. Sympathetic pathways mainly inhibit the bladder by relaxing contraction of the detrusor and simulating contraction of the urinary sphincters. Voiding involves suppression of the sympathetic pathways and stimulation of the parasympathetic pathways which provide excitatory input to increase ureteral peristalsis, cause contraction of the detrusor muscle of the bladder, and relax the sphincters of the bladder neck. The somatic pathways principally innervate the external urethral muscles.

In one embodiment, temporary inhibition of, or reduction in, sympathetic tone is applied to the treatment of urinary hesitancy. The same applies to reducing urinary retention (residual urine in the bladder). Urinary retention has been associated with poor outcomes including urinary tract infections. In one embodiment, local heating is provided in one or more areas of the groin and/or perineum for stimulating the local production of NO which then acts locally and transiently to stimulate contraction of the detrusor muscles of the bladder and to relax the smooth muscle cells of the bladder neck and help further open the neck to reduce urinary hesitancy, increase urinary flow, and decrease the residual urine in the bladder.

The methods for relaxation by heating/warming include using gentle heat increasing in intensity up to the point of maximum tolerance which results in relaxing muscles. In certain embodiments, local heat to the pubic area, groin and/or perineum is provided through a wearable appliance such as a garment that provides local and/or regional heat on demand when the individual wishes to empty their bladder.

In one embodiment, the noninvasive heatable device is dimensioned for placement at a location that will effect heating at a target body to induce local relaxation. The target body can be any sphincter in the body that is innervated by sympathetic nerves, such as for example the bladder neck. In one embodiment the heating method is conventional such as by electric heating coils or is provided by ultrasound, microwave (MW) and/or radio frequency (RF) energy. In particular, in one embodiment ultrasound, microwave (MW) and/or radio frequency (RF) diathermy is employed to generate deep heating up to 2 inches from the skin surface without damage to the skin. Ultrasound diathermy applies high-frequency acoustic vibration to tissues, while MW diathermy applies a strong electrical field with comparatively low magnetic-field energy to induce intra-molecular vibration of highly polar molecules within the treated tissue to generate a thermal effect. RF diathermy involves application of shortwave length, high-frequency electromagnetic fields. The electromagnetic field can be perpendicular or longitudinal in orientation. Although perpendicular electromagnetic field devices have been historically utilized in medical RF diathermy devices, devices able to low-energy longitudinal fields are also available (i.e. Selicor Brand Selitherm devices) and are applicable to the present invention.

In one embodiment of the invention, the heating is provided by Far Infrared Radiation. Commercially available versions of such elements able to provide heat to subcutaneous tissue include, for example, Far Infrared (FIR) Radiant Heating elements. (Challenge Carbon Technology Co., Taiwan). Such elements are suited for FIR heated clothing due to their flat form and foldable, durable and washable properties. The elements as provided for use in clothing include lithium-ion batteries, temperature controller and OCP (Over-Charge Protector) integrated in one controller that provides for rapid heat up according to set upper levels.

In one embodiment of the invention heating is electromagnetic based and is effected by selectively heating a component of the device such as a metal compound. Alternatively the component may be a compound sensitive to heating by ultrasound, such as for example, polymers selectable by those of skill in the art to heat up preferentially by ultrasound. The polymer will preferably have the characteristic that its acoustic impedance exceeds that of surrounding tissue thus preferentially heating the polymer component. Examples of useful polymers in this regard include silicone, polyvinylchloride, nylon, polyurethane and combinations thereof that optimize the heating rate or to useable properties such as flexibility and stability. In this embodiment, one or more layers or inserts of the polymer component are heated by a remote or local ultrasound transducer.

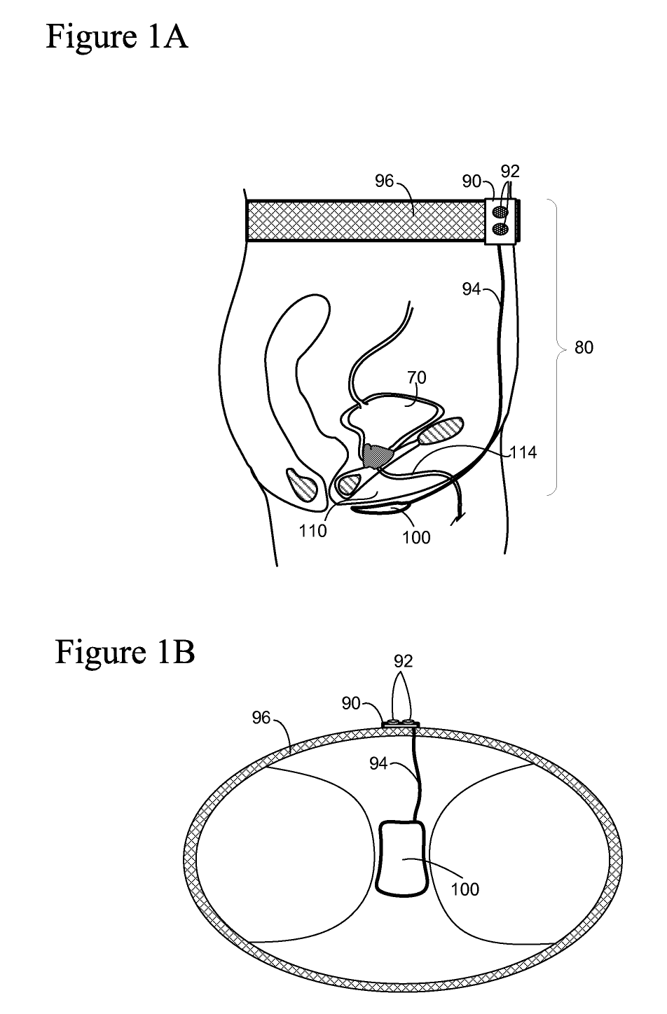

In the case of heating the groin area, and as depicted in FIGS. 1A and B, the wearable product 80 can be heating underwear or a device that can be placed in ordinary underwear. Wearable product 80 can include a power supply 90 and on-off controls 92. The controls 92 and power supply 90 are connected to heating element 100 by one or more leads 94. Warming is controlled and increases progressively until it reaches a threshold of tolerance or the desired effect is obtained. The device controller provides for maximum temperature settings such that a patient can turn the device on and warming will increase progressively until the maximum is reached and will hold steady at this temperature until turned up, down or off.

In another embodiment, underwear type garments are provided that include a pouch dimensioned to hold a heatable insert. The insert can be heated by a heating gel, iron oxide or other materials that produce heat by chemical reaction and which can emit heat for a length of time. Alternatively, the insert can be heated by microwave exposure such as in a microwave oven. When voiding of the bladder is desired or other parasympathetic stimulation, the insert is heated and placed into the pouch to provide warming of the lower abdomen over the bladder and thereby parasympathetic relaxation of the muscles of the bladder.

FIGS. 1A and B depicts one embodiment of a heating device wherein heating element 100 is dimensioned for placement against the perineum 110 of the patient such that heat generated affects tissues near the urethra 114 and thereby provokes a parasympathetic release of NO with attendant relaxation of smooth muscle of the urethra such that emptying can occur from the bladder 70. In one embodiment, the device is worn in association with, or disposed in, underwear, which as used herein includes garters, stockings, athletic supporters and girdle type garments.

The device located therein can heatable via combustible energy sources such as butane or propane heaters, electric energy, electromagnetic energy (infrared radiation). Power can be delivered through a wearable power supply and cause heat on demand.

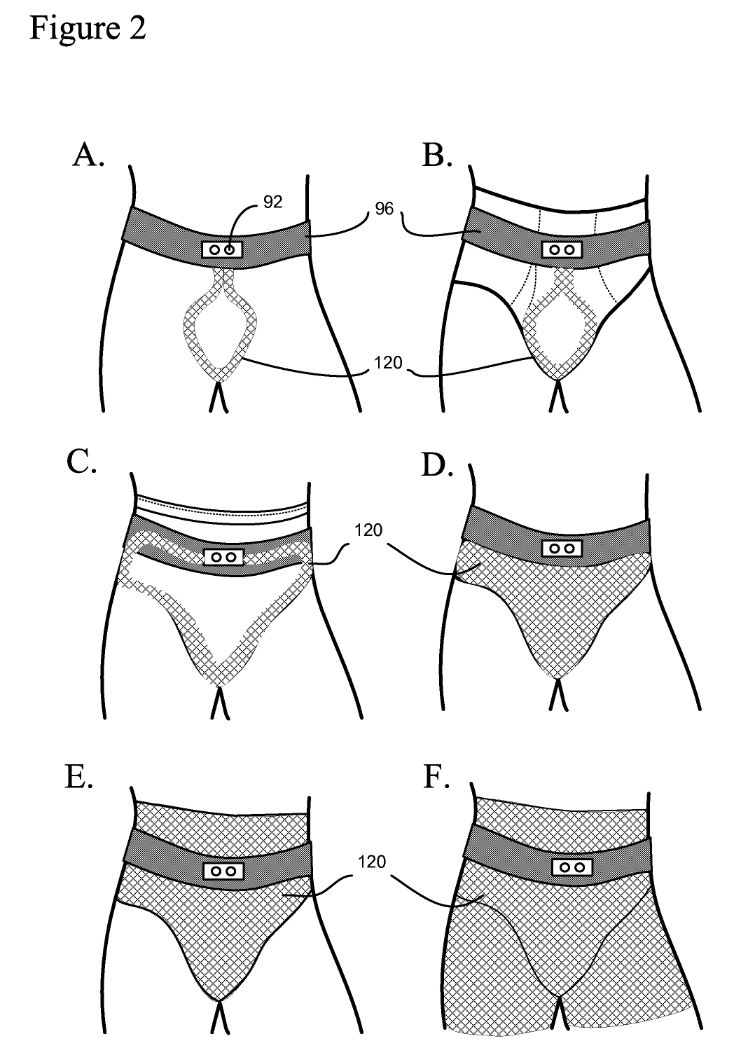

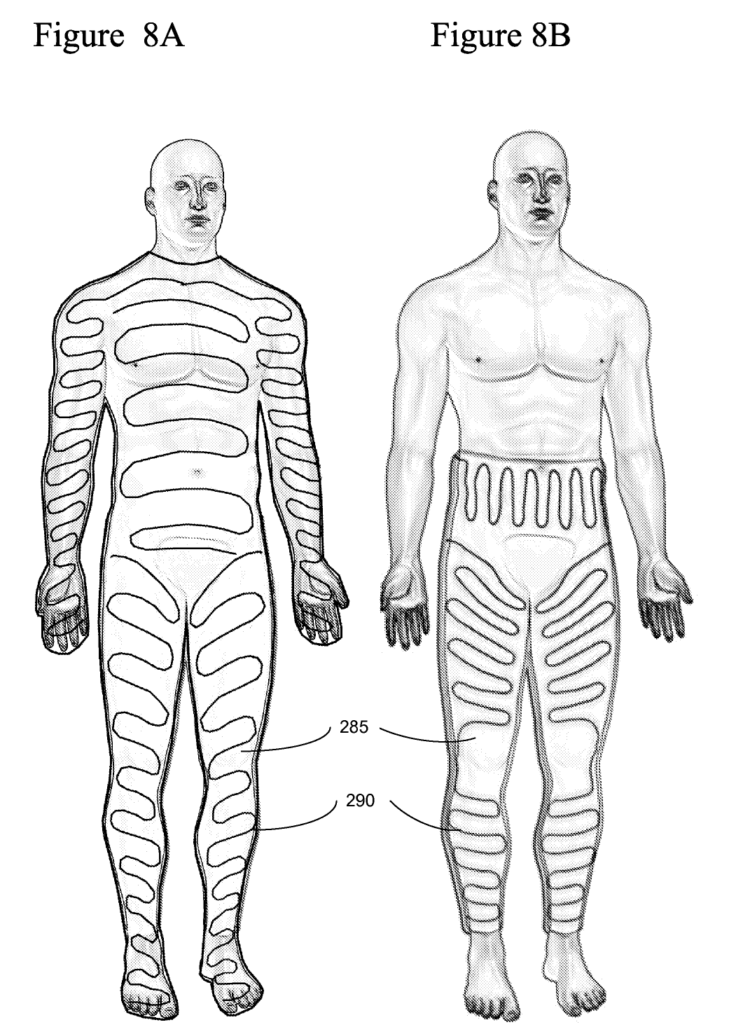

A variety of other wearable devices are envisioned in accordance with the present invention including, for example, embodiments depicted in FIG. 2. In FIGS. 2A and B, devices particularly suitable for male patients are provided that avoid application of heat to the testes. The device can be a standalone device as in FIG. 2A, or can be incorporated into a garment as in FIG. 2B. Other of the embodiments of Figures C-F may be used in either males or females. The garment can be provided with various openings as may be desirable. In each of the embodiments of FIG. 2, the heatable area is depicted with crosshatching 120. In each of the depicted embodiments, local heat to the groin and/or perineum is provided through the wearable appliance such as a garment that provides local and/or regional heat on demand when the individual wishes to empty their bladder. In one embodiment, a form of wearable device can include a belt or an elastic band around stomach that can have a cosmetic function as well. In one embodiment, an over-underwear elastic wearable heating cloth is provided that is composed of a stretchable mesh. Wearing the heating cloth over regular underwear provides certain sanitary advantages. FIGS. 8A and 8B depict further embodiments of heatable pants and shorts wherein heating elements are affixed to the fabric of the garment.

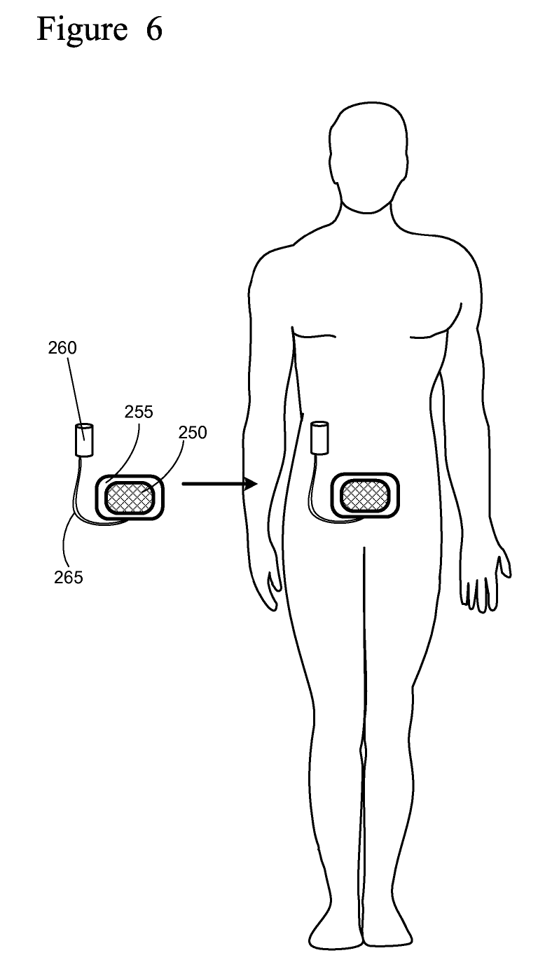

One embodiment adapted for delivery of local heat over lower abdomen is depicted in FIG. 6. Pad 255 includes a heating element 250 that is connected via cable 265 to a controllable power source 260, which is adaptable to be affixed or hung from a belt or other garment around the waist or placed in a garment pocket. Preferably the power source is a rechargeable battery. In one embodiment, the heating element is a FIR element. Optionally, the pad is covered with a heat reflecting or insulating material on the outer surface so that the heat is directed inwards towards the body. In one embodiment, the pad is covered on the outer part by an adhesive layer around the outer edges to stick to the skin of the body. This adhesive outer layer is disposable while the heating pad itself is reusable.

In other embodiments, a systemic parasympathetic response is provided by heating at sites of the body that are removed from or “distal to” the area of the body in which parasympathetic relaxation of smooth muscle is desired. Distal heating provided, for example to the hands or areas thereof, stimulates a reflex parasympathetic response that transits through the central nervous system and back down to reduce sympathetic signaling and provide a parasympathetic stimulus for contraction of the detrusor muscles of the bladder and to relax the smooth muscle cells of the bladder neck and help further open the neck to reduce urinary hesitancy, increase urinary flow, and decrease the residual urine in the bladder. In one embodiment the application of distal heat is provided by wearable products and appliances that may include garments having embedded heating elements such as heating underwear, gloves, socks, shoes, helmets and jackets as well as heatable inserts or pads that are dimensioned for placement in any of the above garments.

In one embodiment, the heating elements embedded in wearable garments and/or inserts are designed for: 1) placement based on the sympathetic/parasympathetic map on the body such that heat is concentrated on local and regional areas to modulate autonomic nervous system to relax targeted muscles, as opposed to generalized heating of the whole body, and 2) is on-demand and aimed at short term local relaxation rather than general systemic relaxation.

In one embodiment of the invention, the distal heating apparatus is a glove or portable handwarmer. The device can be dimensioned for carrying in a pocket or purse. The heating applied to the distal area must be of sufficient magnitude to cause the same increase in parasympathetic response as could be obtained by local heating. The optimal site for heating, as well as the intensity and duration of heating, can be readily determined for a given individual based on whether or not the desired relaxation is obtained.

In other embodiments, local heat sources are adapted to be applied to a desired body part such as a wrist, hand, foot, forehead, ears, and/or nose. In certain embodiments the local heat sources contain a heating gel, iron oxide or other materials that produce heat by chemical reaction and which can emit heat for a length of time. Alternatively, the heat sources comprise a material that is amenable to excitation by microwave exposure such as in a microwave oven. In another embodiment, a local heat source is disposed within a pocket in a stress relief type ball that is designed to be squeezable. The provision of the pocket allows the ball to be reused while the chemical heating element is disposable and can be replaced.

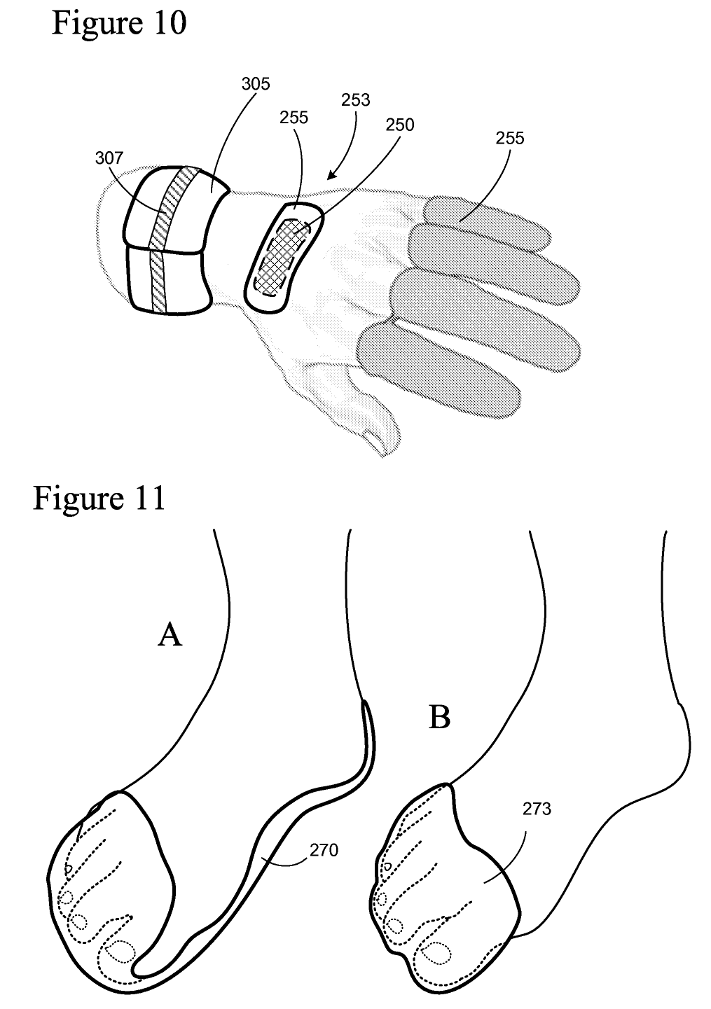

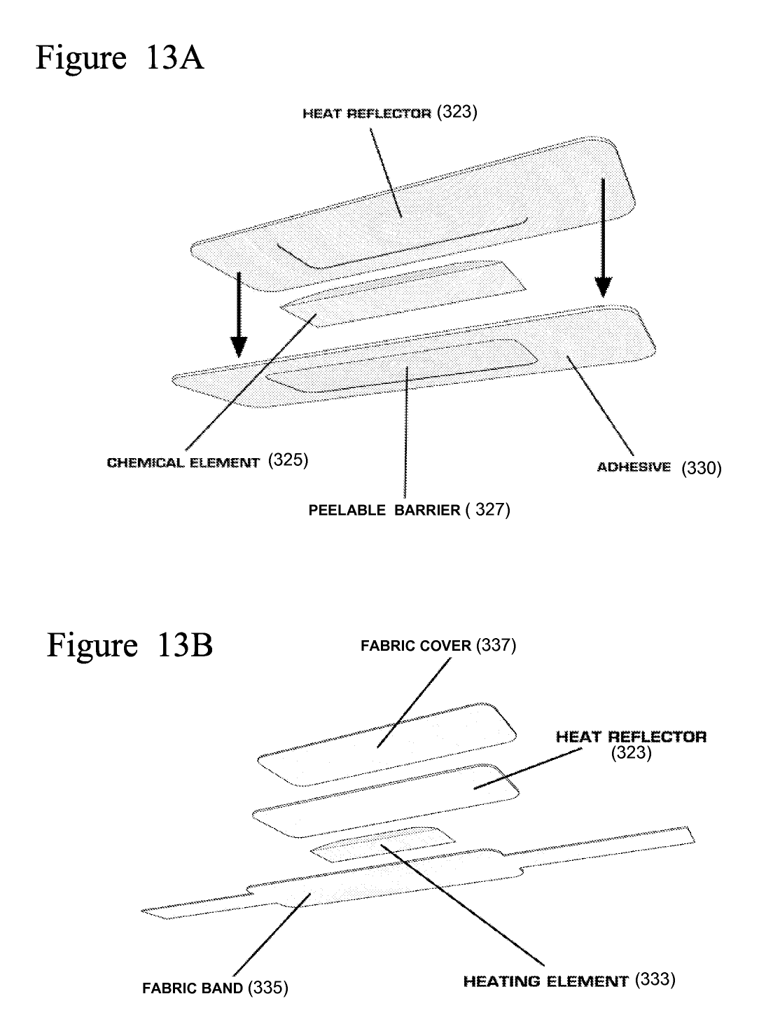

In certain embodiments, such as depicted in FIG. 10, the heat source is dimensioned as heating wrap 305 that has within its shape a flexible metal or similar bending frame material 307 which can form to the shape of the body part it is flexed around. Similarly, the heating nose patch 315 of FIG. 12A includes heating gel/iron oxide/heating chemicals that are in an enclosure dimensioned in the shape the human nose and including bending frame 307 to accommodate different nose sizes and shapes. The inner surface/layer of the patch is covered with a thin layer of plastic adherent which upon separation will produce chemical reactions and generate heat. To increase the efficacy of heat, this product is insulated on the outer side by heat reflector to direct the heat inward. Alternatively, material forming a constituent layer of the heating wrap or nose patch is differentially susceptible to microwave excitation and can be heated by microwave exposure such as in a microwave oven.

In further embodiments also depicted in FIG. 10, disposable heating elements for the hands or fingers are provided. The heating elements may be disposed in full gloves or mittens or as the partial coverings of heating half gloves or finger cots 255. The heat source is a heating gel, iron oxide, or other materials that produce heat by chemical reaction and which can emit heat for a length of time. Alternatively, material forming a constituent layer of the gloves, finger cots or sole inserts is differentially susceptible to microwave excitation and can be heated by microwave exposure such as in a microwave oven. The heating components are molten or formed in the shape of a glove or finger covering portion thereof. The inner surface/layer of the glove or portion thereof is covered with a thin layer of plastic adherent which upon separation will produce chemical reactions and generate heat. To increase the efficacy of heat, the gloves or finger cots are insulated on the outer side by heat reflector to direct the heat inward.

Other embodiments depicted in FIGS. 1A and B, provide heating toe covers 273 or toe covers including a sole 270. FIG. 12A further depicts examples of heating elements 250 located in a head band or cap. The entire band can be heated or elements can be alternatively situated for heating one or more of the forehead, ears and nape of the neck.

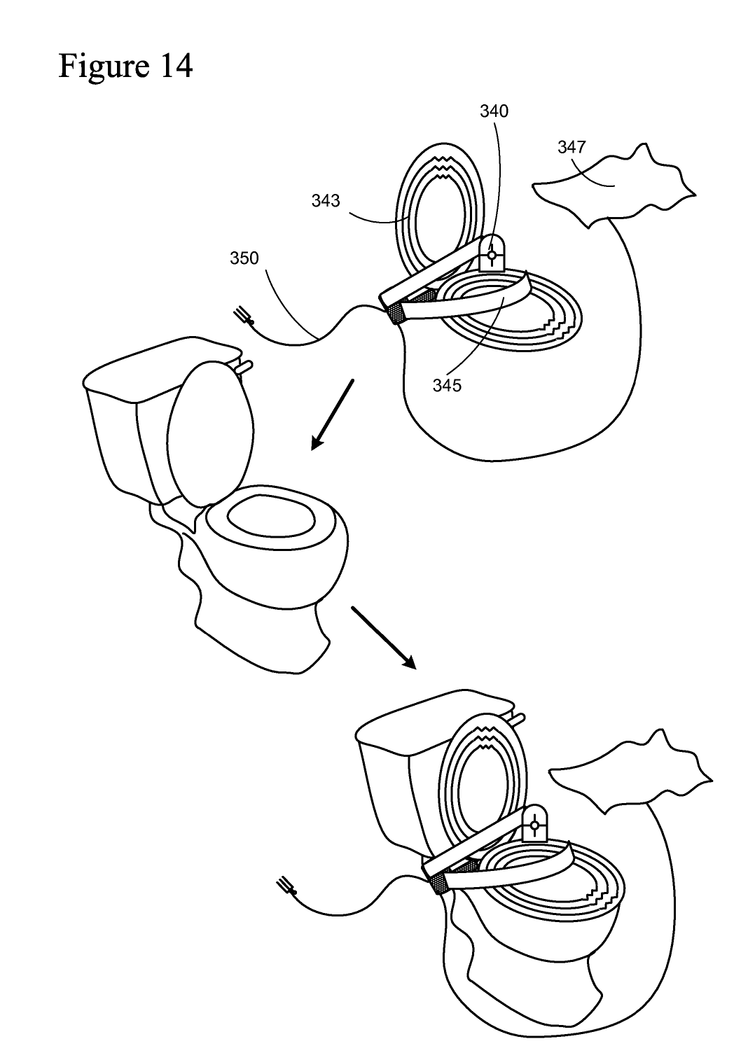

In other embodiments such as that also depicted in FIG. 10 an adhesive heat patch 253 is provided wherein the heat source 250 is provided together with an adhesive layer 255 for affixing the patch to a desired body part. One embodiment of such a patch is depicted in FIG. 13A, wherein an adhesive heat patch is provided wherein the heat source (chemical element 325) is disposed in a patch that is affixed to the desired body part by adhesive layer 330. As depicted in one embodiment in FIG. 13A, the reactive components of the heat source are covered by a thin covering such as peelable barrier 327 whereby separation of the covering would activate the heating process. In the embodiment depicted in FIG. 13A, the chemical heating element 325 is insulated on the outer side by heat reflector 323 to direct the heat inward and thereby increase the efficacy of heating. In the embodiment depicted in FIG. 13B, the heat source is affixed to the desired body part by fabric band 335 which can be connected by any number of closures including, without limitation, hook and loop closures, snaps, buttons and the like.

In certain embodiments, the heat source (such as heating gel, iron oxide, or other reactive exothermic compounds) is adapted to have time release qualities by controlled exposure to air. The control mechanism is provided by layers of insulators that prevent initiation of the exothermic reaction. In one embodiment the layers of insulation of peelable and upon each peel and exposure, a certain amount of heat is produced till the last layer is peeled and the remaining energy is used up.

In another embodiment, heating is available from non-wearable appliances. For an example involving fixtures that located in the bathroom, heated hand holds or hand grips are provided for immediate delivery of heat to the hand including the palm and/or fingertips. The heated hand holds are situated to be reachable from an individual using the toilet. In one embodiment, the plumbing for a public bathroom is designed such that the hot water running in bathroom pipes is directed in a serpentine manner through hand holds in proximity to each toilet. Depending on the temperature of the building hot water, the hand holds may be covered with a thin insulating layer to avoid a dangerously hot surface temperature. In another embodiment, provision of heat is quickly on-demand through valves in the case of fluid heat sources and via switches in the case of electric heat sources. It is anticipated that heaters suitable for use in private places will not be limited by the need to make use in apparent to third parties.

In another embodiment, heat is delivered by an air blower or infrared lamp that delivers local heat to a person to stimulate a parasympathetic response and relaxation of the smooth muscle of the urinary neck. In one embodiment, an air blower is mounted on the wall of a bathroom or bathroom stall and is adapted to direct warm air to a lower abdominal area of a person desiring to relax sufficiently for urination. The air blower may be associated with a wand or flexible tube that can be pulled out and direct warm air where it is needed. In one embodiment the temperature of the warm air is controllable for maximum parasympathetic effect in the individual.

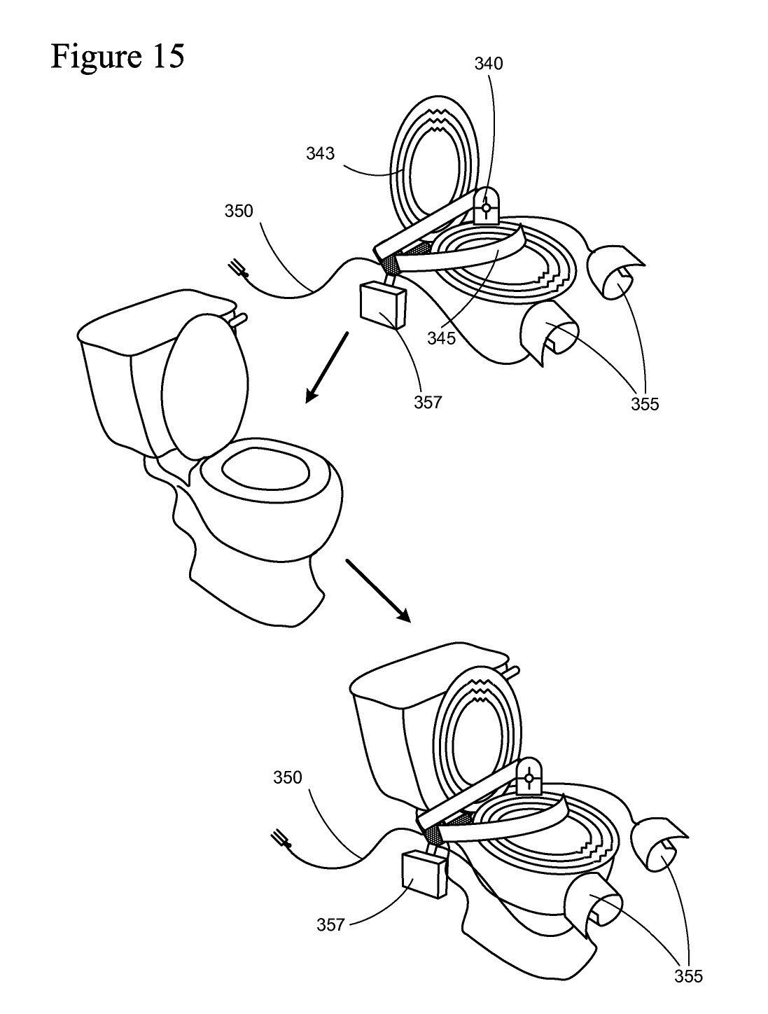

Alternatively as depicted in FIG. 14, the air blower or infrared lamp 340 is part of a toilet system and is aimed to deliver heat to the groin region. In another embodiment, heat is provided locally through heating elements attached to the toilet and designed to direct heat to the groin, pubis and/or perineum when activated individually by the individual in need of parasympathetic stimulation.

In one embodiment of the invention a device and method is provided of heating certain parts of toilet and adding auxiliary heat inducing components. The device has several heating zones; each capable of heating separately or in conjunction with others. The amount of heat can be modulated by a switch. To increase the efficacy of heat, this product may be optionally insulated on the outer side of any included heat source to direct the heat inward. The potential heating zones are heating toilet seat, heating toilet cover (lid), infrared heating lamp, heating belt and heating blanket. Where a lamp is employed it is attached to the toilet seat with an arm capable of directing the lamp and its focus on any general direction desired by the user. The amount of heat can be modulated by a modulating device attached to the side of the lamp, making it capable of dimming or increased intensity.

As depicted in FIG. 14, in one embodiment heating elements 343 circle either or both of the toilet seat and toilet lid. Heat provided by touching a warm surface such as seating on a seated toilet seat is referred to here as heat by conduction or conductive heat. Provision of heating elements on the toilet lid provides heating to the lower back and roots of the nerves innervating the bladder. In one embodiment, the heating system further includes heat wrap 345 dimensioned for laying across the abdomen while seated. In a further embodiment, the heating system further includes heat blanket 347 dimensioned for laying across the thighs of the individual while seated. The components of the combined system including heated toilet seat and/or lid, infrared or warm air blower 340, heat wrap 345 and heat blanket 347, can be supplied individually in modular fashion or as a combined unit that affixes to a toilet and plugs into house current via power cord 350.

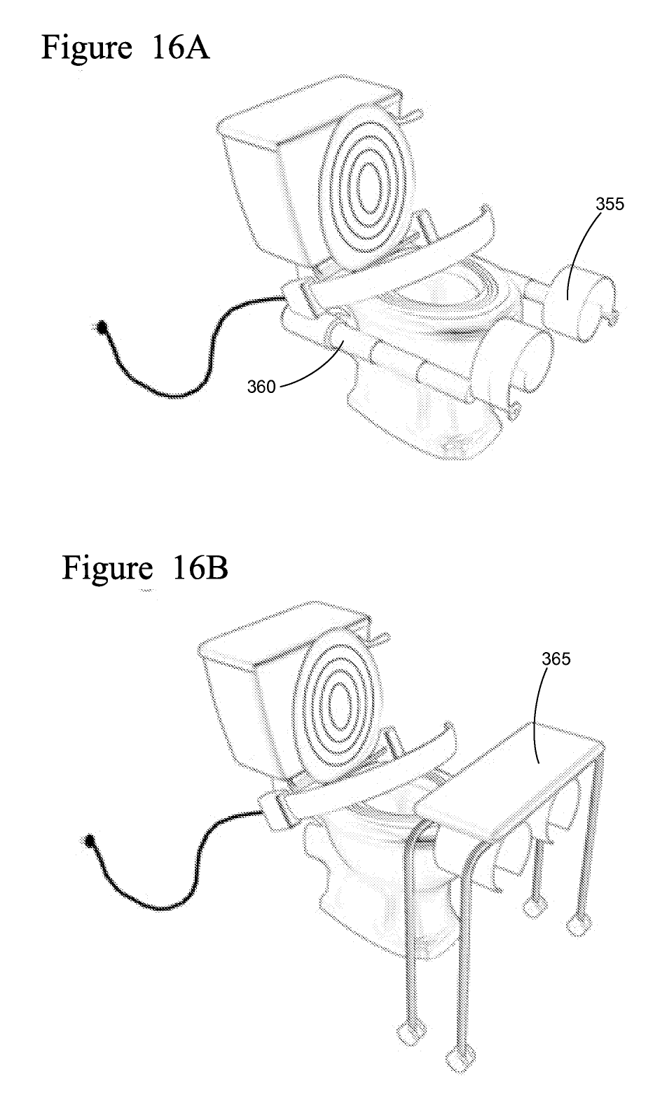

In an alternate embodiment depicted in FIG. 15, in lieu of a heat blanket, heating thigh wraps 355 are provided. The wraps can be stored in one or more holsters 357 attached to a side or sides of the toilet seat system. The wraps are provide modulated heat and can be heated separately or in combination with the other.

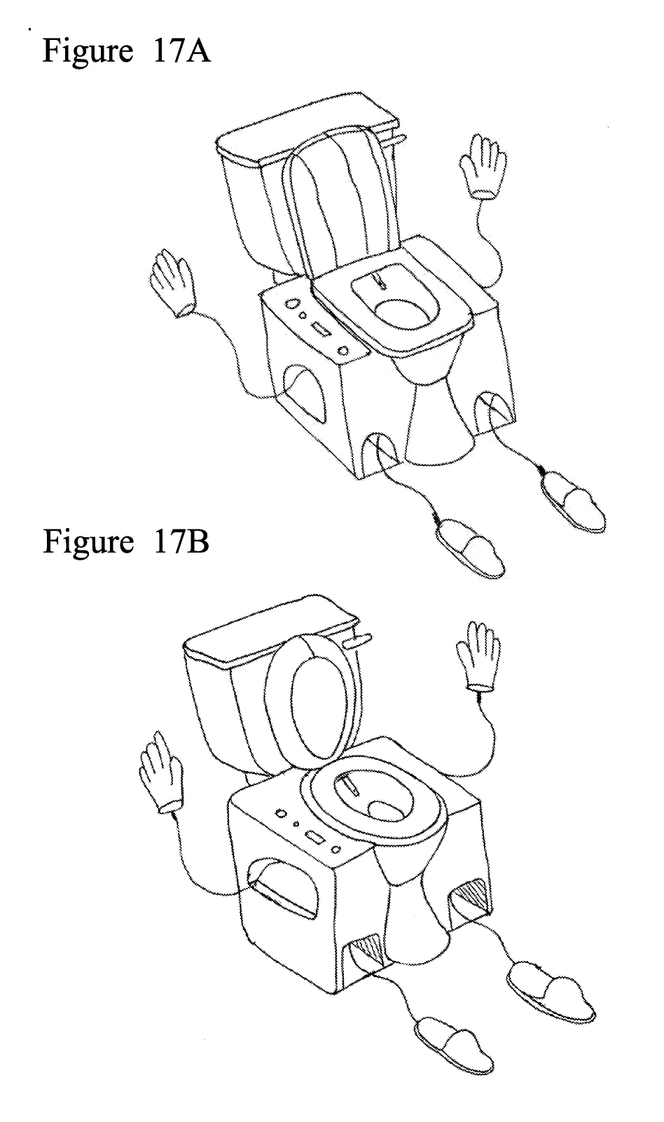

In an alternate embodiment depicted in FIG. 16A the thigh wraps are attached to two fold in/out mechanisms (handle bars 360) located at either side of the toilet seat. When pulled forward the handle bars carry the thigh wraps forward closer to the thigh region. The wraps are heat modulated and can be heated separately or in combination with the other. In a further embodiment depicted in FIG. 16B, a roller type device 365 is provided with thigh wraps attached to it. The device has four wheels and a tray on the upper section. The device is foldable for convenient storage and easy mobility. The thigh wraps are attached to the mid section of the device with a holster type place for storage. The thigh wrap table can be independently powered. The thigh wrap table can be used together with or apart from other heating elements.

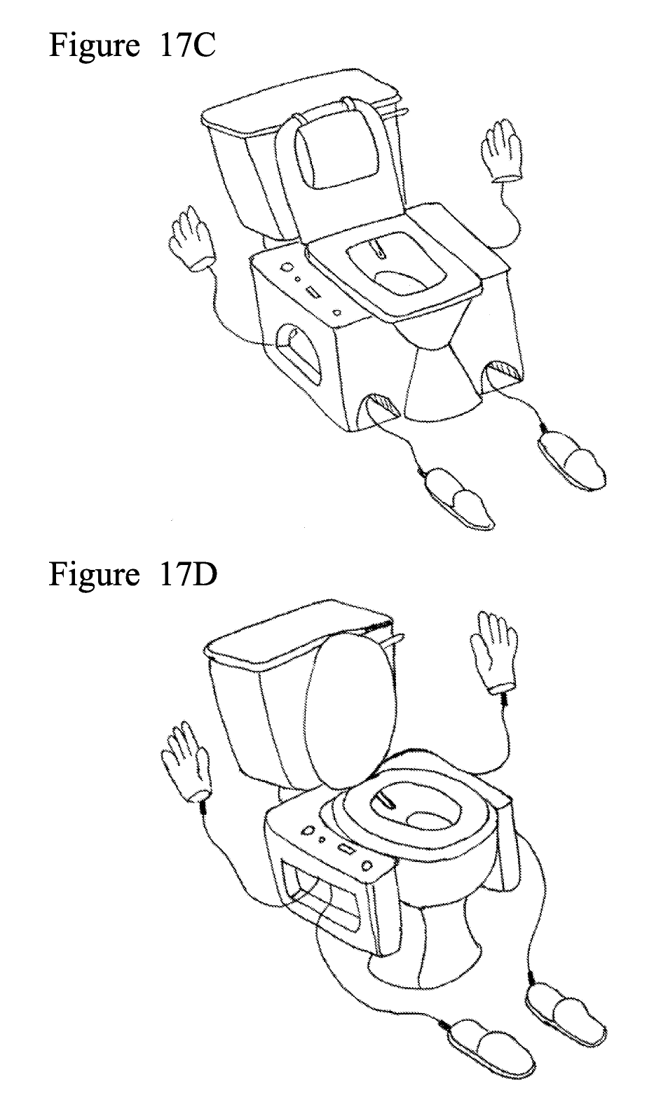

FIGS. 17A-D depict further embodiments of a toilet systems having heating components designed for parasympathetic stimulation. The systems have several heating zones; each capable of heating separately or in conjunction with others. The amount of heat can be modulated by a switch. The heating zones include: heating toilet seat, heating toilet cover (lid), heating gloves, and heating socks/slippers. Optionally, the toilet system may be accessorized by a hot water line around the interior of the bowl, which is capable of infusing hot water when needed. Optionally, the system includes an element capable of releasing an “aroma therapy” odor/fragrance when desired in order to stimulate parasympathetic activity and create relaxation.

In another embodiment, infrared heaters and/or warm air blowers are situated in proximity to the toilet to direct heat to the hands, face, neck and/or torso with sufficient heat and force to induce a systemic parasympathetic effect by distal heating. The temperature and force should not be so aggressive as to induce an adverse sympathetic response. The heating apparatus in the bathroom has to be designed in such a way to minimize any adverse or associated factors that can create or exacerbate anxiety and induce sympathetic surges. For example, heating with a hot-air-blower that is noisy can make it obvious to third parties that the individual is suffering from hesitancy or SBS, the thought of which can worsen the situation for the individual. In light this principle, multiple designs are envisioned for public bathrooms using IR heat radiation, electric heaters, and heated hand holds that can be turned on and off without raising awareness in the surrounding area.

In one embodiment, whether local or distal administration of heat is chronic, regular or periodic for sustained reduction of intraluminal pressure of the bladder neck. For example, the individual patient may schedule a pattern of heating, such as for limited periods 5-10 times a day for reduction of intraluminal pressure of the bladder neck, in the treatment of urinary hesitancy, urinary retention and in Shy Bowel Syndrome. Depending on responses desired and obtained in the individual patient, the intensity and duration of heat can be tuned for optimal responses.

In another embodiment, parasympathetic stimulation is delivered through inhalation, such as inhalation of warm air. Certain areas in certain people can be more sensitive. For example heating facial skin may result in a more prominent effect than heating an arm in certain individuals and the delivery heat can be tested in various locations and then subsequently applied where most efficacious.

In one embodiment, a mask type device 320 such as that depicted in FIG. 12B is placed over the mouth and the nose area to provide parasympathetic stimulation or reduce sympathetic activity. Disposed with the mask is a heat source 250 wherein the heat is generated by the action of compounds such as heating gels, iron oxides or other chemical compounds that generate heat by reaction such as by oxidation. When breathing through the mask, the heat exchange of inhaled cold air through the mouth and over the heat source creates warm air by the simple process of breathing in.

For distal heating the duration of heating will typically be longer to arouse a systemic response than local heating in the groin, public and perineal areas. In both situations the intensity has to increase up to the comfort boundaries and should not rise to the level where it becomes a stressful stimuli that triggers sympathetic activity.

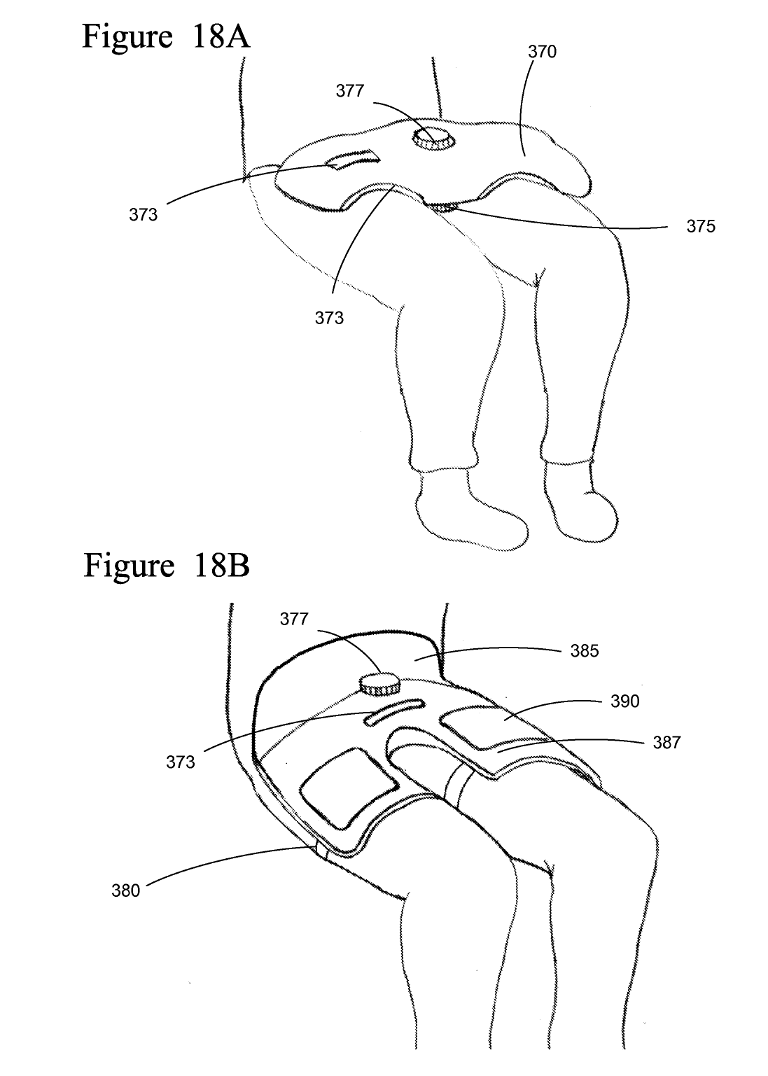

In one embodiment of the invention depicted in FIG. 18A, a heating bag adapted to provide parasympathetic stimulation or reduce sympathetic activity. In one embodiment, the heat bag 370 is adapted to be filled with a warm or hot fluid and placed on the lap when seated. Alternatively the bag may be filled or prefilled with a substance that is differentially heated by microwave radiation relative to the bag construction material. In this embodiment, the bag contents are heated by a microwave source such as for example a microwave oven by placing the entire bag in a microwave. In one embodiment depicted in FIG. 18A, the bag is dimensioned to have underside indentations 373 for both thighs. In one embodiment, the bag has two openings, a top filling opening 375 used to pour the hot water in and a bottom drain opening 377 to evacuate the bag. The bag is further provided with a thermometer 380 to gauge the outside temperature of the bag. In a preferred embodiment the thermometer is a liquid crystal strip thermometer.

In an alternative embodiment, depicted in FIG. 18B, a heating bag is provided that adopts a horse-shoe or inverted U type configuration including thigh extensions 387 to at least partially cover each upper thigh region while leaving access to the genital region. Optionally, the heating bag further includes pockets 390 over each thigh for insertion and warming of the individual's hands. In one embodiment the bag is provided with leg straps 380 that may be closed around the leg by a closure such as a hook and loop type closure. In one embodiment, a heating bag is provided that includes a heatable belly flap or extension 385 for providing heat to the lower abdomen. The belly flap may include pockets that warm the hands as the hands hold the flap tightly against the abdomen. In one embodiment, the heating bag is provided in conjunction with one or more elements of the toilet system described herein.

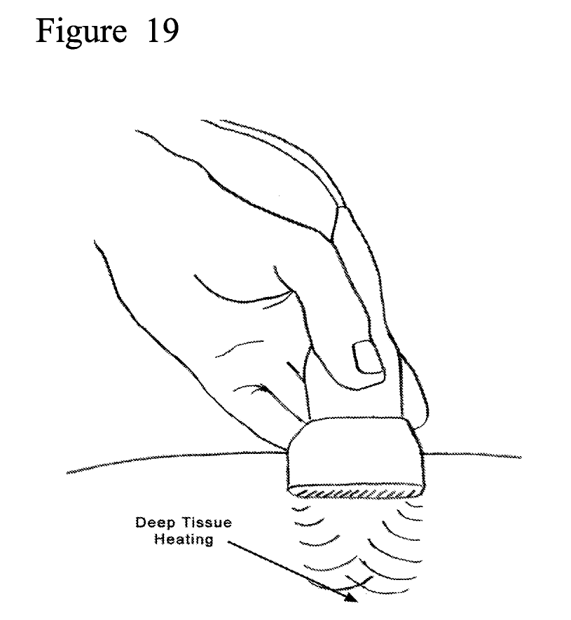

In one embodiment of the invention depicted in FIG. 19B, deep tissue heating is provided using ultrasound. This method of heating uses a trans-cutaneous or trans-abdominal heating of certain parts of the body namely the bladder neck area to facilitate the relaxation of the sphincter muscles for the purpose of aiding urination.

Treatment of DESD:

In some patients with spinal cord injuries, particularly suprasacral injuries, when bladder emptying is attempted, the urinary sphincter contracts along with the bladder. This is termed detrusor-external sphincter dyssynergia (DESD) and results in failure of bladder emptying, often resulting in bladder infection as well as high bladder pressure which can result in hydronephrosis and loss of kidney function. Alpha blockers have not been useful in reducing bladder pressure in DESD and there remain no effective drug treatments. The only surgical treatment is sphincterotomy, which is associated with significant problems and is often ineffective. Delivery of NO by oral or sublingual administration or delivery of NOS encoding genes to the muscle cells of the urethra has been proposed in the treatment of DESD. Mamas M A, et al. Nitric Oxide and the Lower Urinary Tract: Current Concepts, Future Prospects. Urology 61 (2003) 1079-85. In one embodiment of the present invention, heat is utilized to stimulate the local release of NO in the neck of the bladder and proximal urethra in the treatment of DESD.

Treatment of Shy Bladder Syndrome:

In another indication, the application of heat is designed for acute effects in treatment of shy bladder whereby sphincter relaxation and detrusor contraction is induced through exposure of the skin to tolerable heat to help with urination. Avoidant paruresis can start at any age and affects mainly boys or men, although girls and women can also suffer from it. It has been estimated that one in ten Americans suffers to some degree from “bashful bladder” syndrome, the chronic inability to use a public bathroom when nature calls. The disorder, which appears to run in families, ranges in severity. In mild cases, the affected individual requires the use of a stall instead of a urinal. Moderate cases are able to relieve themselves only in a stall and when the bathroom is empty, while severe cases are unable to urinate no matter how private the surroundings. As a result, the disorder can force many otherwise healthy people to become practically housebound. In some instances, sufferers have bought homes close to their work so they could go home whenever they needed to urinate. Existing therapies include social conditioning and drug therapy with sedatives, anti-anxiety drugs and antidepressants, as well as with alpha-adrenergic blockers and with parasympathetic nervous system stimulants such as bethanechol.

In one embodiment of the present invention, either local or distal heating is employed to lower the resistant threshold that prevents urination. Use of heat therapy for reducing sympathetic surge as disclosed here can be used alone or in combination with existing anti-anxiety therapies as needed.

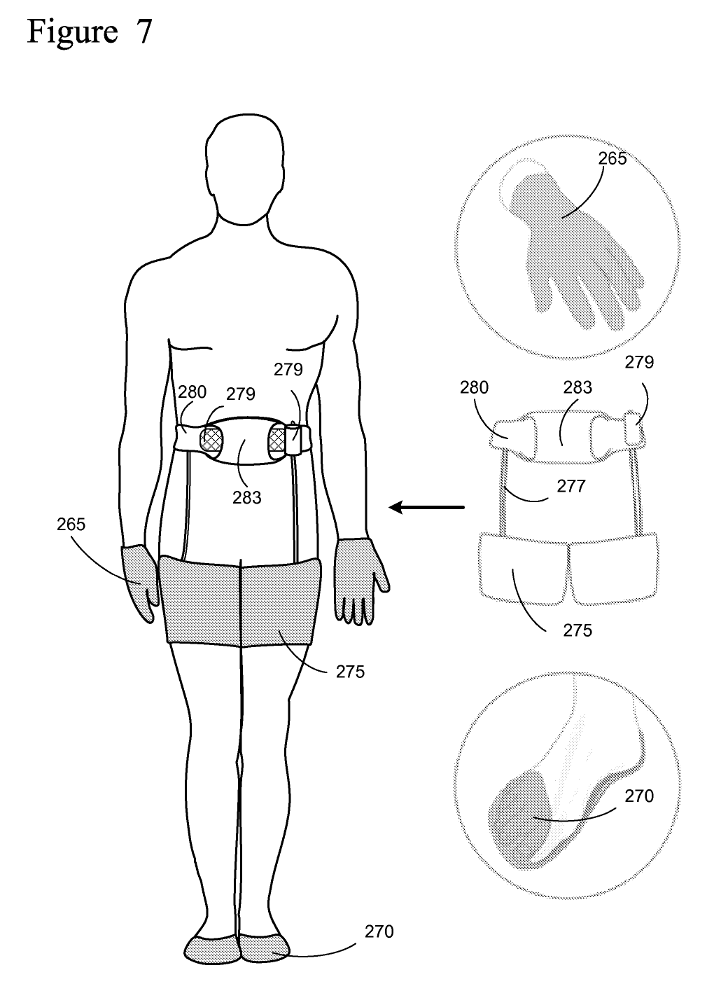

One ambulatory embodiment adapted for treatment of shy bladder/BPH or nocturia is depicted in FIG. 7. As depicted, a shy bladder/BPH package is provided including two heatable thigh pads 275 and a belt 280 with a heatable frontal pocket 283 for hand insertion. When not in use the thigh pads may be rolled and packed in the pocket. The entire belt can be heated or a portion thereof, such as the depicted embodiment wherein heating element 279 is disposed behind within the pouch to heat hands placed in the pouch. The thigh pads are connected to the belt via cables 277 and the source for heating power is a rechargeable battery 279 located on the belt. In one preferred embodiment, elements used for heating are far infrared ray (FIR) heating material. The thigh pads and the belt may be heated jointly or independently per user desire. To increase the efficacy of heat, optional insulation on the outer side such as by a far infrared ray heat reflective material directs the heat inward. In one embodiment, the shy bladder/BPH package further includes heating gloves 265 and or shoe inserts 270 connectable to the power source.

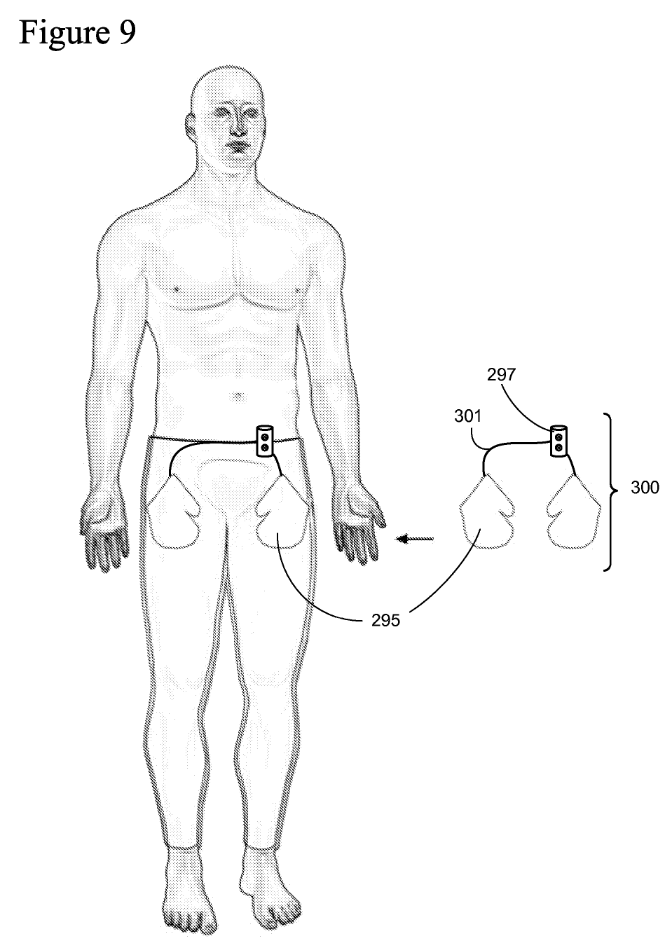

In another embodiment for treatment of shy bladder/BPH/nocturia depicted in FIG. 9, a heating pocket insert package 300 is provided. The package includes at least two frontal pockets 295 for hand insertion, each electrically connected via cables 301 to a controllable power source 297, preferably a rechargeable battery. The heating pocket inserts are dimensioned to be can be conveniently and inobtrusively tucked in the front pockets of the wearer. Optionally, the package may further include a belt for mounting of the controllable power source. In one embodiment, the heating elements used for heating are the far infrared ray heating material. Optionally, to increase the efficacy of heat, the pockets are heat insulated on the side disposed away from the skin to direct the heat inward. When voiding is desired, the user discretely turns on the power source allowing the pocket inserts to heat up. In one embodiment, the inserts are dimensioned to accommodate the hands of the user such that the warm pockets can be situated and pressed against the abdomen for maximal heating.

Local Heating for and by Insertable Medical Devices:

In one embodiment of the invention, a local heating device for use in hospital settings is provided for modulation of parasympathetic balance for insertion of medical devices through sphincters. For example, heat is applied to the lower abdomen to induce a parasympathetic relaxation of the urinary sphincter prior to insertion of a catheter into the bladder. Alternatively, the device itself may be designed to deliver local heating, such as for example a heating aspect associated with urinary tubes. Heating induces relaxation of the sphincter such that reduced force is required to pass the device through the sphincter. Thus, there is reduced risk of perforation and the procedure causes less discomfort to the patient. In one embodiment, heat is employed locally or regionally to relax sphincter muscles. For example, for insertion of devices including Foley type catheters, cytoscopes, and stone removal devices, into the bladder or up into the urethra, heat is applied to the groin and lower abdomen prior to insertion of the device. In other embodiments, the device itself is heated or heatable. In one embodiment for the assisting in the insertion of medical devices, heating is provided by diathermy.

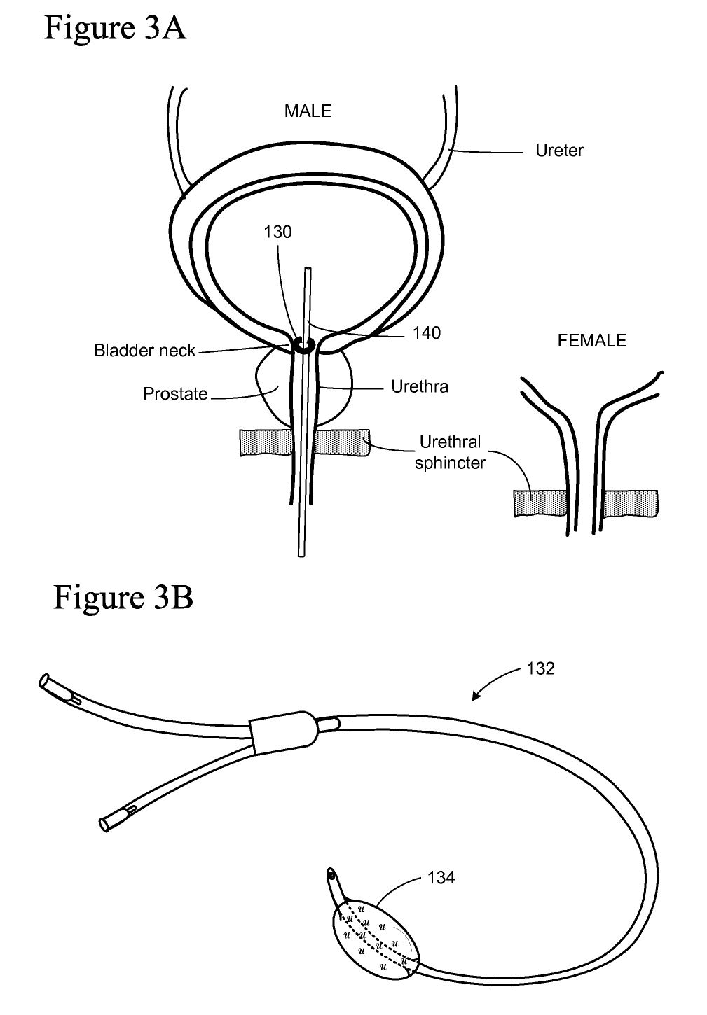

In one embodiment, a device is dimensioned to deliver local heating from inside a fluid passageway such as the urethra and or the ureter, such as for example a heating aspect associated with urinary tubes. In other embodiments, transdermal, transluminal, or local radiation is delivered, including focused heat such as ultrasound or microwave heat. In one embodiment, heat is delivered through radiation but the target organ includes a substance or device for differential absorption of the heat/radiation. For one non-limiting example, in the case of urinary tubes or cystoscopes, a small metal ring or band 130 of material that can be differentially heated is embedded in or on the tube or catheter 140 at a location where it will engage the relevant sphincter such as for example the neck of bladder as shown in FIG. 3A. The heatable material is heated noninvasively by RF or electromagnetic radiation that is absorbed by the metal or other differentially heatable compound such as a polymer. The implanted device may alternatively include one or more layers of a polymer composition that is sensitive to heating by ultrasound. Certain polymers that have the property of being preferentially heatable include silicone, polyvinylchloride, polyurethane, nylon, phosphorylcholine and combinations thereof that may optimize the heating rate of the coating or to improve stability or biocompatibility of the coating.

In another embodiment, a differentially heatable implant is dimensioned for insertion and placement proximal to the bladder, such as against a detrusor muscle, the bladder wall or the neck of the bladder is provided. In one embodiment, local heating to the bladder and surrounding tissues of the female lower abdominal anatomy is provided by a heated intra-vaginal probe inserted in the vagina. The heat is of a level sufficient to increase parasympathetic activity and induce relaxation of muscles in the vaginal vault as well as adjacent tissues in the rectal and bladder regions. The heat can be generated by several means including electrically, chemically, or by infrared radiation, microwave or ultrasound vibration. In one embodiment, the probe or implant heats differentially in response to RF, electromagnetic, ultrasound or microwave radiation from an external source.

In one embodiment, the heat or radiation source is activated when emptying of the bladder is desired and the local heating stimulates a parasympathetic response that allows for emptying of the bladder. Once again the heat level must avoid that which would be sufficiently stressful as to elicit sympathetic surges. The level of stress is determinable by the subject's comfort zone. In any event, the local temperature elicited must be less than what would cause tissue injury or irreversible damage.

In another embodiment as depicted in FIG. 3B, a balloon catheter 132 is provided wherein the balloon 134 is designed to receive an inflation fluid that includes one or more chemicals or reactive compounds U that generate heat from the inflated balloon. The heating balloon can be disposed on many different catheter designs including “Foley” type catheters of several designs including without limitation Coudé elbowed catheters, Council tip catheters having provision for a guidewire and 3-way catheters having a further arm over that depicted in FIG. 3B. In an alternative embodiment, the catheter is manufactured with one or more chemicals disposed within the balloon wherein the chemicals react to generate heat upon contact with a standard inflation fluid such as saline. In one embodiment, the generated heat is in the range of about 38° C. to about 44° C., which is sufficient to reduce inflammation by causing selective apoptosis of inflammatory cells as disclosed in U.S. Patent Application Ser. No. 2002/0193785, Naghavi et al.

Pre-Treatment Assessment of Conditions:

In one embodiment, methods and apparatus are provided for evaluating the intensity of sympathetic over-reactivity. In one example, a multi-channel heat monitoring device continuously probes heat at the fingertips and/or toes before, during, and after a sympathetic stimulator such as, for example, a mental challenge test or a cold exposure. This device and method distinguishes the hyper-adrenergic component of hesitancy in males who might have both BPH and spastic (hyperactive) bladder neck.

Monitoring Effects of Treatment for Urinary Hesitancy:

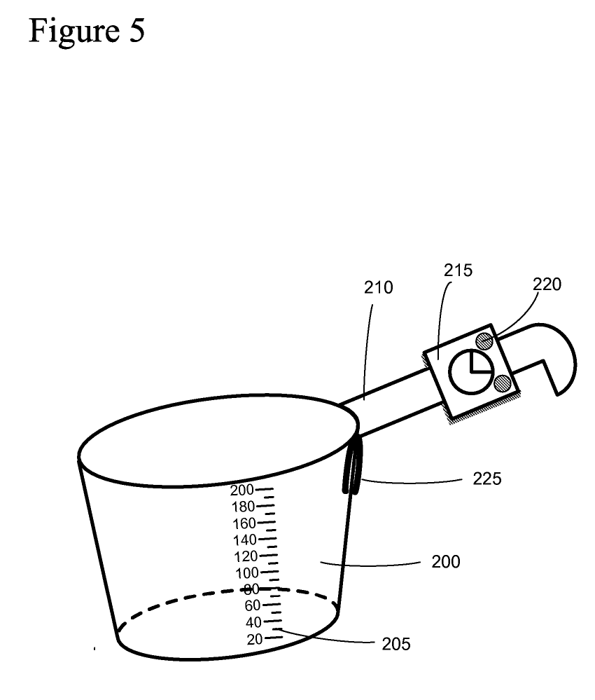

In one embodiment, an example of which is in FIG. 5, a urine collection container 200 marked with volume scales 205 is provided together with a timer, such as a stopwatch type of timer, allowing the user to measure his or her urinary output flow by volume over time. As depicted, an oval or oblong container shape with a handle 210 may be desirable for collection in a sitting position. The handle may be mounted with a timer 215 having on and off buttons 220 that are pressed when the person starts and stops voiding as well as a display of elapsed time. The total volume over time is used as a measure of functional obstruction at the bladder neck. In one embodiment, a disposable or reusable plastic container to collect urine is configured with a handle 210 for conveniently holding the container in position. Where a disposable or otherwise removable container is desired, the handle may be configured with a clip 225 for affixing the handle to the container. Alternatively, where the disposable container is flexible, such as a bags that are dimensioned for collection and volume measurement of urine, the handle may include a ring that holds the bag. Different designs for collection by males and females may be employed in accordance with anatomical variation.

In one embodiment, a kit is provided for quantitative self evaluation of efficacy of a treatment for urinary hesitancy, the kit including a graduated container for measuring urination volume, a timing device having a start button and a stop button, and a log for recording urination volume over urination time. Such a kit is particularly suitable for evaluating the results of treatment in clinical trials of new therapies for urinary conditions.

Treatment of Nocturia:

In one embodiment of the invention, methods and apparatus for treatment of nocturia (voiding during the night) are provided. Nocturia is a common problem in the elderly who have high residual volume in their bladder after voiding. In accordance with the present invention, local or distal heating of sufficient intensity and duration to induce a parasympathetic response is employed before going to bed in order to reduce the residual urine in the bladder and thereby reduce the frequency of voiding during the night. In one embodiment, diathermy is employed to relax the neck of the bladder and to stimulate contraction of the bladder to facilitate full emptying. In one embodiment, the heating device is installed in the home bathroom such that it can be readily employed prior to going to bed. Also more specifically, this method and apparatus is of particular advantage in individuals who experience a significant adverse effect due to pharmacologic treatments of hesitancy and frequency such as hypotension, dry ejaculation, etc.

Treatment of Laryngeal Spasm:

Laryngeal spasm is a persistent contraction of the larynx muscles (voice box) such that the vocal cords to come together. The spasm can result in partial or complete blockage of the entrance to the trachea (windpipe). In one embodiment of the invention, noninvasive use of heat, including local, regional and systemic (reflex based) heat, is use to reduce the tone or spasm of laryngeal muscle. The local heating can be administered by a device either self mounted on the neck by the patient for intermittent or chronic use, or as fitted in position by a medical professional. The heat can be focused or concentrated for deep tissue heating (like with ultrasound or microwave) deep enough to reach the muscles (1-3 inches).

Manipulation of the Carotid Sinus Reflex:

In one embodiment, the non-pharmacologic modulation of the ANS involves methods and apparatus for placing controlled pressure on the carotid to manipulate the carotid sinus reflex, which plays a central role in blood pressure homeostasis. Baroreceptors associated with the carotid sinus or bulb, which is located at the bifurcation of the internal and external carotids, are designed to detect changes in stretch and transmural pressure. Such changes are registered by afferent impulses that are transmitted by the carotid sinus nerves to nuclei in the brain stem. In response, efferent impulses are carried through sympathetic and vagus nerves to the heart and blood vessels, controlling heart rate and vasomotor tone. Carotid massage is used by physicians to diagnose Carotid Sinus Hypersensitivity (“CSH”), a condition in which mechanical deformation of the carotid sinus causes exaggerated bradycardia or vasodilatation responses, resulting in hypotension, presyncope, or syncope. Carotid massage also used as a bedside technique by physicians for non-pharmacologic rapid treatment of tachy-arrhythmias such as paroxysmal atrial tachycardia (PAT). In one embodiment of the invention, the carotid clip is used to provide controlled pressure on the carotid sinus in the diagnosis and management of CSH.

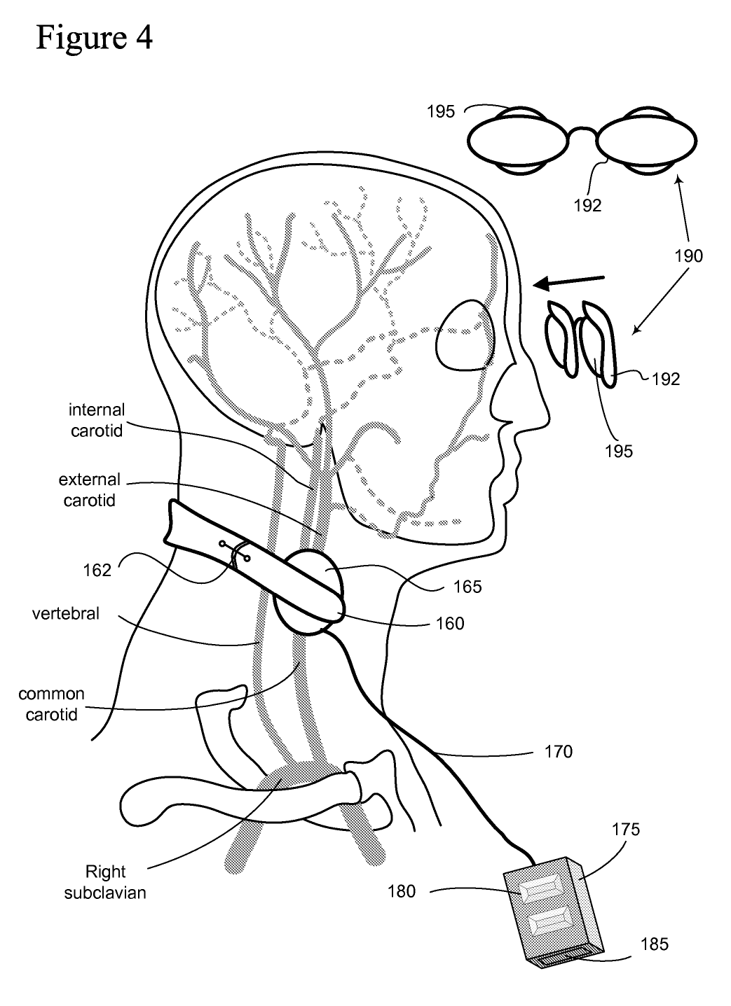

One embodiment of the invention, the carotid sinus reflex is exploited in order to provide a method for modulating the ANS to favor the parasympathetic and thereby induce smooth muscle relaxation in the treatment of a number of disorders including urinary retention. For one example as depicted in FIG. 4, a neck clip or band 160 is provided that includes a pad 165 that is dimensioned to create external pressure, and optionally massage and/or heat, on the carotid sinus. The clip can optionally provide for adjustment via a slide mechanism 162 such that the clip or band can be fitted to accommodate the dimensions of the patient's neck size and the individual location of the carotid bifurcation. For delivery of heat and vibration, the pad 165 is connected via a lead 170 to a controller 175 that includes on-off switches such as toggle switches 180. For devices including heating and vibration capability, the controller may include a battery 185 or may alternatively provide for connection to an external source of power such as an AC source. Prior to dispensing the device, the patient is tested using the device to insure that the patient is free of CSH, for which the device would be contraindicated.

The patient is educated to place the stimulator at the location of the maximal carotid impulse, medial to the sternomastoid muscle at the upper border level of the thyroid cartilage. Pressure, heat and vibration can be gauged and controlled by such a device. The purpose of the device is to standardize the technique of placing increased pressure at the carotid sinus to shift the ANS balance towards the parasympathetic but without inducing asystole or a dangerous reduction of blood pressure. However, by allowing the patient to control the placement, pressure, degree of heating and vibration, the required stimulation is provided. In one embodiment, the device is further provided with a blood pressure monitor and may include an alarm if blood pressure drops below a preset level.

Ocular Massage:

In another embodiment, ocular massage is employed to increase parasympathetic tone. Ocular massage is a procedure that involves applying pressure (compression) on the eyes and, like carotid message, is employed to stop PAT. In one embodiment, an eyepiece such as eyepiece 190 depicted in FIG. 4 device is provided that includes a frame 192 dimensioned to fit over both eye sockets and to support eyepads 195 provide gentle pressure, with or without heating, as needed to stimulate a parasympathetic response and thereby enable urination or defecation. The eyepads 195 can be solid such as foam or can be filled with a variety of materials known in the art. In one embodiment, the eyepads are filled with a fluid or gel that can be heated for use such as in a microwave. The device can be conveniently carried in a purse or pocket until needed.

Treatment of Heart Failure:

In other embodiments, heat is used to treat heart failure where increased production of nitric oxide is needed. In this embodiment, long term usage is envisioned for providing generalized or systemic vasodilation by shifting the sympathetic-parasympathetic balance and increasing local production of nitric oxide. The applied heat is delivered below the individual's tolerance or comfort level and is applied slowly and increased in accordance with individual comfort such that the application of heat does not cause stress or exceed a person's tolerance, in which case a sympathetic surge and overtone may be provoked. In one embodiment for the treatment of heart failure, a heating garment can be employed as often as desired, for example, several times a day on a manual or timed schedule.

The invention provides heating garments including underwear, vests and body suits, and wearable heating accessories like gloves and socks for heart failure patients who are in a vasoconstrictive mode, which is a well known stage of heart failure. Vasoconstriction caused by excess sympathetic activity in turn causes a failing heart to pump harder against a higher vascular resistance. As evidence of this, heart failure patients typically have cold fingers (extremities). Specialized heating devices are provided that can enable partial or total body heating on a controlling degree of heating and timing cycle that avoid the stress of too much heat. In one embodiment, an astronaut type cloth garment is provided that allows the patient to control the heat at different spots with different temperatures. FIGS. 8A and 8B depict embodiments of heatable whole and partial body suits wherein heating elements 290 are affixed to the fabric of the garment 285. Further smaller partial suits are also envisioned such as heating abdominal wraps and heating shorts.

If desired, the amount of heat can be titrated by monitoring blood pressure or by brain natriutic peptide levels (BNP) a well known serum marker of response to therapy. The heat is controllable to be administered slowly and should not increase heart rate beyond a level where it crosses individual's tolerance and increases significantly the activity of cardiovascular system. In one embodiment, a pulse monitor is included with the device such that the heating cycle is turned off or down when a predetermined decrease in heart rate is reached.

Other Local Heat Treatment Indications

In certain embodiments, methods are provided for selective modulation of the ANS by application of heat to areas of the body that afford particularly sensitive access to the ANS such as the fingers, and areas of the face including the ears. Local application of heat can be useful not only in medical indications but also to provide comfort and safety in cold environments. FIG. 12B depicts several such embodiments including a heating mask 320 as well as adhesive ear patches 321 and ear lobule clips 322. In such embodiments, heat may be generated by the action of integral compounds such as heating gels, iron oxides or other chemical compounds that generate heat by reaction such as by oxidation. In such chemical reactive heaters, the reactive components of the heat source are covered by a thin covering such as peelable barrier whereby separation of the covering activates the heating process. Alternatively, the heat source can be reusable such as by inclusion of materials that are selectively excited microwave radiation. Whether the heat source is a chemically reactive heating element or is reusable and heated by micromave, the element can be optionally insulated on an outer side by a heat reflector that directs the heat inward and thereby increases the efficacy of heating.

The mask 320, ear patches 321 or lobule clips 322 are applied either to provide modulation of the autonomic system or for increased comfort and safety when the face and ears are exposed to cold temperatures such as in cold working or sports environments. Alternatively, the ear patches or lobule clips may be used to provide palliative support for painful conditions such as ear infections. The ear patch 321 is dimensioned to cover a portion or all of the exposed ear surface. The ear lobule clip 322 is dimensioned to be applied around the ear lobe or lobule. The ear patch and/or lobule clip may be adhered to the surface of the ear or portions thereof by an inner surface adhesive or by included flexible metal or similar bending frame materials which can be formed around the individual ear pinna and/or lobule.

In another embodiment (not depicted), adhesive heating nails are provided that are dimensioned to cover all or a portion of one or more finger or toe nails. The heating nails can be provided with one time chemical reaction heating elements or can be reusable by having included materials that are selectively heated by microwave. Such heating nails may be useful medically either to provide modulation of the autonomic system or for local heating of digits that may be affected by circulatory compromise such as, for example, by injury, diabetes, atherosclerosis or Raynaud's phenomena. Alternatively, the heating nails may be used for increased comfort and safety when the fingers are exposed to cold temperatures such as in cold working or sports environments.

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

We claim:

1. An apparatus for control of bladder function comprising a controlled heating device that facilitates voiding of the bladder when desired.

2. The apparatus of claim 1, wherein the heating is applied prior to voiding to reduce sympathetic activity and prepare the bladder for voiding.

3. The apparatus of claim 1, wherein the device is wearable.

4. The apparatus of claim 1, wherein the device is worn in association with, or disposed in, underwear.

5. The apparatus of claim 1, wherein the device comprises a pair of heating pocket inserts.

6. The apparatus of claim 5, wherein the heating pocket inserts are dimensioned to enclose a hand.

7. The apparatus of claim 1 wherein the heat is provided by diathermy directed to the bladder area.

8. The apparatus of claim 1, wherein the heating device generates heat by chemical reaction or is heated by microwave action.

9. The apparatus claim 1, wherein the heat is provided in treatment of lower urinary tract symptoms such as urinary hesitancy, nocturia, and shy bladder conditions.

10. An apparatus for control of bladder function, comprising:
at least one heating element disposed in a wearable appliance that applies heat to a body region distal to the bladder when voiding of the bladder is desired.

11. The apparatus of claim 10, wherein the region is one or more of: a portion of the head, hands, feet and thighs.

12. The apparatus of claim 10, wherein the appliance is disposed in, or in association with, one or more garments selected from the group consisting of: gloves, socks, shoes, helmets, caps, head bands, wrist bands, scarves, jackets, vests, and abdominal wraps.

13. The apparatus of claim 10, comprising a plurality of heating elements including at least heating elements that apply heat to at least the hands, feet and lower abdominal regions simultaneously.

14. The apparatus of claim 10, wherein the appliance comprises a pair of heating thigh bands.

15. The apparatus of claim 14, further comprising a heating hand pocket dimensioned to be worn around the waist.

16. The apparatus of claim 10, wherein the heating element generates heat by chemical reaction or is heated by microwave action.

17. An apparatus for control of bladder function in an individual, comprising:
a heat source disposed in a non-wearable fixed or portable appliance that controllably delivers heat to the individual when voiding of the bladder is desired.

18. The apparatus of claim 17, wherein the appliance delivers conductive heat, radiant heat, warm air, and/or a warm hand hold.

19. The apparatus of claim 17, wherein the heat source is a heating bag that is dimensioned to conform to a shape of the upper thigh surface.

20. The apparatus of claim 17, wherein the heating bag is U-shaped.

21. The apparatus of claims 19 or 20, wherein the heating bag further comprises a heating belly flap.

22. The apparatus of any one of claims 19-21, further comprising a thermometer, such as a liquid crystal thermometer, affixed to the heating bag.

23. The apparatus claim 17, wherein the heat is provided in treatment of one or more of urinary hesitancy, shy bladder and nocturia conditions.

24. A fixture for controllably applying heat locally, regionally and/or distally relative to a bladder area of an individual, the fixture comprising:
at least one heating element located as part of a toilet system, whereby the heating element delivers heat sufficient to facilitate voiding of the bladder.

25. The fixture of claim 24, wherein at least one heating element is a heating wrap dimensioned to provide heat to a lower abdominal region.

26. The fixture of claim 24, wherein at least one heating element is a heating blanket dimensioned to provide heat to the upper thighs.

27. The fixture of claim 24, wherein at least one heating element is a pair of thigh wraps dimensioned to provide heat to the upper thighs.

28. The fixture of claim 24, wherein at least one heating element is a heating toilet set lid.

29. The fixture of claim 29, wherein at least one heating element is a pair of slippers dimensioned to provide heat to the feet.

30. A balloon catheter comprising a balloon dimensioned to receive an inflation fluid that includes or activates one or more chemicals or reactive compounds that generate heat from the inflated balloon.

31. An intra-vaginal probe that comprises a heating element.

32. The intra-vaginal probe of claim 31, wherein the heating element generates heat by chemical reaction or comprises a compound that is activated by microwave radiation to emit heat.

33. A method for control of bladder function in treatment of urinary hesitancy, shy bladder and nocturia conditions in a patient, comprising:
supplying at least one heating appliance that is dimensioned to apply local heat to at least one area of the patient to facilitate voiding of the bladder when desired.

34. The method of claim 33, wherein the area is selected from the group consisting of the lower abdomen, lower back, perineum, upper thighs, feet, hands, and combinations thereof.

35. The method of claim 34, wherein the appliance comprises a pair of heating thigh bands.

36. The method of claim 34, wherein the appliance comprises a pair of heating hand pockets or pocket inserts.

37. The method of claim 34, wherein the appliance is worn in association with, or disposed in, underwear.

38. The method of claim 33 wherein the heating appliance includes a controller that is operable by the patient for turning on and off of the heating element.

39. The method of claim 33, wherein the area is one or more of: face, fingers, and toes.

40. The method of claim 33, wherein the appliance is disposed in, or in association with, one or more of: gloves, socks, shoes, helmets, caps, head and/or ear bands, ear patches, ear lobe clips, nail covers, wrist bands, scarves, jackets, vests, and abdominal wraps.

41. A method for control of bladder function in an individual, comprising:
turning on a heat source disposed in non-wearable fixed or portable appliance that delivers heat locally and/or distally to the individual when voiding of the bladder is desired; and
turning off the heat source when voiding of the bladder is completed.

42. The method of claim 41, wherein the appliance delivers radiant heat, warm air, or a warm hand hold.

43. The method of claim 41, wherein the heat source generates heat by its effect in tissue by diathermy.

44. The method claim 41, wherein the heat is provided in treatment of one or more of urinary hesitancy, shy bladder and nocturia conditions.

45. A method for quantitative self evaluation of efficacy of a treatment for urinary hesitancy comprising:
determining a baseline urinary output flow by measuring urination volume over urination time;
initiating treatment; and
determining a series of treatment urinary output flow by urination volume over urination time measurements; and evaluating the treatment objectively based on improvements in urination volume over urination time.

46. A kit for quantitative self evaluation of efficacy of a treatment for urinary hesitancy comprising:
a graduated container for measuring urination volume; and
a timing device having a start button and a stop button and a display of elapsed time, and a log for recording urination volume over urination time.

47. The kit of claim 46, wherein the container comprises a handle for conveniently holding the container in position.

48. The kit of claim 47, wherein the timing device is mounted on the handle.

49. The kit of claim 47, wherein the container is disposable and the handle is removable from the container for reuse.

50. A method for shifting a sympathetic-parasympathetic balance to treat of heart failure, comprising applying sufficient heat provided by a wearable appliance or garment that includes an on-off controller to intermittently deliver sufficient heat to result in peripheral resistance.

51. The method of claim 50, wherein the garment is selected from the group consisting of: underwear, gloves, socks, shoes, helmets, scarves, jackets and vests.

52. A method for shifting a sympathetic-parasympathetic balance to dilate a sphincter to facilitate passage or placement of a medical device comprising;
applying heat sufficient to induce dilation of the sphincter; and
inserting the device through the dilated sphincter or placing the medical device in the sphincter.

53. The method of claim 52, wherein the heat is applied regionally.

54. The method of claim 53, wherein at least a portion of the medical device is heated.

55. The method of claim 52, wherein the heated medical device is dimensioned for passage through, or placement in, the bladder neck.
